(19) United States
(12) Patent Application Publication
(10) Pub. No.: US 2010/0111941 A1
(43) Pub. Date: May 6, 2010

(54) METHODS FOR TREATING CANCER

(76) Inventors: Steven Deitcher, San Mateo, CA (US); Jingsong Zhao, Fremont, CA (US)

Correspondence Address: FULBRIGHT & JAWORSKI L.L.P., 600 CONGRESS AVE., SUITE 2400, AUSTIN, TX, 78701, US

(21) Appl. No.: 12/441,320

(22) PCT Filed: Sep. 12, 2007
(86) PCT No.: PCT/US07/78225

§ 371 Date: Mar. 13, 2009

Related U.S. Application Data
(60) Provisional application No. 60/844,551, filed on Sep. 13, 2006.

(51) Int. Cl. A61K 39/395; A61K 38/16; A61P 35/00; A61P 35/04

(52) U.S. Cl. 424/133.1; 514/12

(57) ABSTRACT

The invention relates to the treatment of abnormal or undesirable cell proliferation, particularly endothelial cell proliferation related to tumor growth. The compositions comprise anti-metastatic agents in combination with one or more anti-cancer agent.

This application claims the benefit of priority to U.S. Provisional Patent Application No. 60/844,551 filed Sep. 13, 2006.

### TECHNICAL FIELD OF THE INVENTION

The present invention relates to compositions and methods for treating cancer using anti-metastatic agents in combination with anti-cancer agents.

### SEQUENCE LISTING

The sequences of the polynucleotides and polypeptides of the invention are listed in the Sequence Listing and are submitted electronically in the file labeled “NUVO-29PCT_ST25.txt”-6.81 KB (6981 bytes) which was created on an IBM PC, Windows 2000 operating system on Aug. 31, 2007 at 10:02:10 AM. The Sequence Listing entitled “NUVO-29PCT_ST25.txt” is herein incorporated by reference in its entirety.

### BACKGROUND OF THE INVENTION

Cellular proliferation is a normal ongoing process in all living organisms and is one that involves numerous factors and signals that are delicately balanced to maintain regular cellular cycles. Normal cells divide until maturation is attained and then only as necessary for replacement of damaged or dead cells. When normal cellular proliferation is disturbed or somehow disrupted, the results can affect an array of biological functions. Disruption of proliferation can be due to a myriad of factors such as the absence or overabundance of various signaling chemicals, or the presence of altered environments, or both.

Cancer generally refers to a disease caused by the uncontrolled, abnormal growth of cells in one organ or tissue type that can spread to adjoining tissues or other parts of the body. Cancer cells can form a solid tumor, in which the cancer cells are massed together, or exist as dispersed cells, as in leukemia. Cancer cells are often referred to as malignant because they continuously divide, often crowding out nearby cells and spreading to other parts of the body. The tendency of malignant cancer cells to spread from one organ to another or from one part of the body to another distinguishes them from benign tumor cells, which overgrow but do not spread to other organs or parts of the body. Malignant cancer cells typically metastasize and spread to other parts of the body via the bloodstream or lymphatic system, where they can multiply and form new tumors, making cancer a serious and often deadly disease. Although there have been great improvements in the diagnosis and treatment of cancer over the years, still, many people die each year from various cancers, due to metastases and cancers that are resistant to available therapies. Conventional treatments for cancer include chemotherapy, radiotherapy, surgery, biological therapy (i.e., vaccines or targeted therapeutics) or combinations thereof. However, certain cancers are non-responsive or minimally responsive to treatments such as chemotherapy. Furthermore, some of the available treatments, such as surgery, may not be viable alternatives, depending on the type of cancer.

Once the diagnosis of cancer is established, an important question is whether the disease is localized or has spread to lymph nodes and distant organs. In nearly 50% of patients, surgical excision of primary tumors is ineffective because metastasis has occurred by the time the tumor is large enough for surgical removal (Sugarbaker, Am. J. Pathol. 97:623-32 (1979); Fidler and Balch, Curr. Probl. Surg. 24:129-209 (1987)). Metastatic tumors can be located in several organs and in different regions of the same organ, making complete eradication by surgery, radiation, drugs or biotherapy difficult. Furthermore, the organ microenvironment significantly influences the response of tumor cells to therapy (Fidler, Cancer Res. 45:4714-26 (1985)), as well as the efficiency of anti-cancer drugs, which must be delivered to tumor foci in amounts sufficient to destroy cells without causing undesirable side effects (Fidler and Poste, Semin. Oncol. 12:207-13 (1985)). In addition, the treatment of metastatic cancer is often greatly hindered due to the biological complexity of cancer cells and the rapid emergence of tumor cells that become resistant to most conventional anti-cancer agents (Fidler and Poste, 1985 supra).

One of the many biological processes involved in the growth of both primary and secondary (metastatic) tumors is neovascularization, or creation of new blood vessels that support the growth of the tumor. This neovascularization is commonly termed angiogenesis (Folkman, Cancer Res. 46:467-73 (1986); Folkman, New Eng. J. Med. 320:1211-2 (1989)). Although tumors of about 1-2 mm in diameter can usually receive nutrients by diffusion, further growth typically depends on the development of an adequate blood supply through angiogenesis. Thus, inhibition of angiogenesis may provide a successful approach for decreasing or preventing metastases by interfering with the blood supply to the tumors. Studies in animal models have suggested that the seeding of tumor metastases is a coagulation-dependent process (Palumbo et al., Blood 96:3302-3309 (2000); Donnelly et al., Thromb. Haemost. 79:1041-1047 (1998); Esumi et al., Cancer Res. 51:4549-4556 (1991)). Several studies suggest that the tissue factor/factor VIIa complex may support tumor growth and angiogenesis (Fernandez and Rickles, Curr. Opin. Hematology 9:401-406 (2002); Ruf et al., Immunol. Res. 21:289-292 (2000)). Hembrough et al., Cancer Res. 63:2997-3000 (2003) report that rNAPc2 and TFPI allegedly decrease the growth of lung carcinoma tumors and melanoma tumors in mouse models.

It is an object of the present invention to provide novel compositions and treatments to decrease or inhibit tumor growth or metastasis.

### SUMMARY OF THE INVENTION

The present invention provides methods of preventing or decreasing undesirable endothelial cell proliferation in a subject in need thereof comprising administering an anti-metastatic agent in combination with one or more anti-cancer agent. In a preferred embodiment, the anti-metastatic agent is a NAP protein. In a particular embodiment, the NAP protein demonstrates selective inhibition of Factor Xa. Preferably, the Factor Xa inhibitory NAP protein is selected from NAP5 and NAPE. In a preferred embodiment, the NAP protein demonstrates selective inhibition of the Factor VIIa/Tissue Factor (fVIIa/TF) complex when Factor Xa, or a catalytically active or inactive derivative thereof, is present. More preferably, the fVIIa/TF selective inhibitory NAP protein is selected from rNAPc2 and rNAPc2/proline. Most preferably, the fVIIa/TF selective inhibitory NAP protein is rNAPc2/proline. In a particular embodiment, the anti-cancer agent is a chemotherapeutic agent such as FOLFOX or FOLFIRI or other 5-fluorouracil-derived therapies. In another embodiment, the anti-cancer agent is a radiotherapeutic agent. In yet another embodiment, the anti-cancer agent is a combination of FOLFOX or FOLFIRI and a targeted anti-cancer agent such as a monoclonal antibody (i.e., bevacizumab).

The invention also provides methods of treating cancer in a subject in need thereof comprising an anti-metastatic agent in combination with one or more anti-cancer agent. In a preferred embodiment, the anti-metastatic agent is a NAP protein. In a particular embodiment, the NAP protein demonstrates selective inhibition of Factor Xa. Preferably, the Factor Xa inhibitory NAP protein is selected from NAP5 and NAP6. In a preferred embodiment, the NAP protein demonstrates selective inhibition of the Factor VIIa/Tissue Factor (fVIIa/TF) complex when Factor Xa, or a catalytically active or inactive derivative thereof, is present. More preferably, the fVIIa/TF selective inhibitory NAP protein is selected from rNAPc2 and rNAPc2/proline. Most preferably, the fVIIa/TF selective inhibitory NAP protein is rNAPc2/proline. In a particular embodiment, the anti-cancer agent is a chemotherapeutic agent such as FOLFOX or FOLFIRI or other 5-fluorouracil-derived therapies. In another embodiment, the anti-cancer agent is a radiotherapeutic agent. In yet another embodiment, the anti-cancer agent is a combination of FOLFOX or FOLFIRI and a targeted anti-cancer agent such as a monoclonal antibody (i.e., bevacizumab). In yet another embodiment, the method is used to treat colorectal cancer. In still another embodiment, the method is used to treat melanoma. Further embodiments provide for the treatment of breast, prostate, or lung cancer.

The invention further provides methods of decreasing or inhibiting tumor metastasis in a subject in need thereof comprising administering an anti-metastatic agent in combination with one or more anti-cancer agent. In a preferred embodiment, the anti-metastatic agent is a NAP protein. In a particular embodiment, the NAP protein demonstrates selective inhibition of Factor Xa. Preferably, the Factor Xa inhibitory NAP protein is selected from NAP5 and NAP6. In a preferred embodiment, the NAP protein demonstrates selective inhibition of the Factor VIIa/Tissue Factor (fVIIa/TF) complex when Factor Xa, or a catalytically active or inactive derivative thereof, is present. More preferably, the fVIIa/TF selective inhibitory NAP protein is selected from rNAPc2 and rNAPc2/proline. Most preferably, the fVIIa/TF selective inhibitory NAP protein is rNAPc2/proline. In a particular embodiment, the anti-cancer agent is a chemotherapeutic agent such as FOLFOX or FOLFIRI or other 5-fluorouracil-derived therapies. In another embodiment, the anti-cancer agent is a radiotherapeutic agent. In yet another embodiment, the anti-cancer agent is a combination of FOLFOX or FOLFIRI and a targeted anti-cancer agent such as a monoclonal antibody (i.e., bevacizumab).

The invention also provides methods of reducing tumor burden in a subject in need thereof comprising administering an anti-metastatic agent in combination with one or more anti-cancer agent. In a preferred embodiment, the anti-metastatic agent is a NAP protein. In a particular embodiment, the NAP protein demonstrates selective inhibition of Factor Xa. Preferably, the Factor Xa inhibitory NAP protein is selected from NAP5 and NAPE. In a preferred embodiment, the NAP protein demonstrates selective inhibition of the Factor VIIa/Tissue Factor (fVIIa/TF) complex when Factor Xa, or a catalytically active or inactive derivative thereof, is present. More preferably, the fVIIa/TF selective inhibitory NAP protein is selected from rNAPc2 and rNAPc2/proline. Most preferably, the fVIIa/TF selective inhibitory NAP protein is rNAPc2/proline. In a particular embodiment, the anti-cancer agent is a chemotherapeutic agent such as FOLFOX or FOLFIRI or other 5-fluorouracil-derived therapies. In another embodiment, the anti-cancer agent is a radiotherapeutic agent. In yet another embodiment, the anti-cancer agent is a combination of FOLFOX or FOLFIRI and a targeted anti-cancer agent such as a monoclonal antibody (i.e., bevacizumab).

The invention also relates to a pharmaceutical composition comprising a combination of an anti-metastatic agent and one or more anti-cancer agent. In a preferred embodiment, the anti-metastatic agent is a NAP protein.

The invention also relates to a composition which comprises an anti-metastatic agent, preferably a NAP protein, and an anti-cancer agent in the presence or absence of one or more pharmaceutically acceptable carrier materials.

The invention also provides for the simultaneous or chronologically staggered administration to a subject in need thereof, within a period of time sufficient for both the anti-metastatic agent and the anti-cancer agent to act in combination, for treating a proliferative disease such as cancer. In a preferred embodiment, the anti-metastatic agent is a NAP protein.

Yet another object of the present invention is to provide compositions comprising an anti-metastatic agent and one or more anti-cancer agent wherein the compositions further comprise pharmaceutically acceptable carriers that may be administered intramuscularly, intravenously, transdermally, orally or subcutaneously.

The invention also provides for kits comprising in a pharmaceutically acceptable form, therapeutically effective amounts of an anti-metastatic agent and one or more anti-cancer agent.

### BRIEF DESCRIPTION OF THE DRAWINGS

FIG. 1: Amino acid sequence of: A)rNAPc2 (SEQ ID NO: 3) and B) rNAPc2/pro (SEQ ID NO: 4).

FIG. 2: Effect of rNAPc2/pro, 5-FU, rNAPc2/pro+5-FU or rNAPc2/pro+5−FU+AVASTIN® (bevacizumab) on xenograft colon tumor volume (white bars) or tumor weight (black bars) represented as percent of vehicle control.

FIG. 3: Timecourse of rNAPc2/pro, 5-FU, rNAPc2/pro+5-FU or rNAPc2/pro+5-FU+AVASTIN® on xenograft colon tumor volume (mm³).

FIG. 4: Timecourse of rNAPc2/pro, 5-FU, rNAPc2/pro+5-FU or rNAPc2/pro+5-FU+AVASTIN® on animal body weight.

FIG. 5: Effect of rNAPc2/pro, AVASTIN®, rNAPc2/pro+AVASTIN® or rNAPc2/pro+AVASTIN®+5-FU on xenograft colon tumor volume (white bars) or tumor weight (black bars) represented as percent of vehicle control.

FIG. 6: Timecourse of rNAPc2/pro, AVASTIN®, rNAPc2/pro+AVASTIN® or rNAPc2/pro+AVASTIN®+5-FU on xenograft colon tumor volume (mm³).

FIG. 7: Timecourse of rNAPc2/pro, AVASTIN®, rNAPc2/pro+AVASTIN® or rNAPc2/pro+AVASTIN®+5-FU on animal body weight.

FIG. 8: Dose response of rNAPc2/pro on the number of lung metastases in mice injected with CT26 colon carcinoma cells.

FIG. 9: Dose response of rNAPc2/pro on lung mass of mice injected with CT26 colon carcinoma cells.

FIG. 10: Effect of rNAPc2/pro, CPT-11 or CPT-11+rNAPc2/pro on the extent of liver metastasis in mice injected with HCT116 human colorectal cancer cells.

FIG. 11: Effect of rNAPc2/pro, CPT-11 or CPT-11+rNAPc2/pro on liver wet weight in mice injected with HCT116 human colorectal cancer cells.

### DETAILED DESCRIPTION OF THE INVENTION

Headings used herein are for ease of reading and are not to be construed as limiting the description of the invention in any way.

### I. DEFINITIONS

Unless otherwise defined herein, scientific and technical terms used in connection with the present invention have the meanings that are commonly understood by those of ordinary skill in the art. Further, unless otherwise required by context, singular terms include pluralities and plural terms include the singular. Unless otherwise indicated, nucleic acids are written left to right in 5′ to 3′ orientation; amino acid sequences are written left to right in amino to carboxy orientation.

Standard techniques are used for recombinant DNA, oligonucleotide synthesis, and tissue culture and transformation (e.g. electroporation, lipofection). Enzymatic reactions and purification techniques are performed according to the manufacturer's specifications or as commonly accomplished in the art or as described herein. The foregoing techniques and procedures are as generally performed according to conventional methods well known in the art and as described in various general and more specific references that are cited and discussed throughout the present specification. See Sambrook et al., Molecular Cloning: A Laboratory Manual, 2d ed., Cold Spring Harbor Laboratory Press, Cold Spring Harbor, N.Y. (1989), Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990), which are incorporated herein by reference in their entirety for all purposes. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The terms “a,” “an” and “the” mean one or more and include the plural unless the context is inappropriate.

The terms “polypeptide” or “protein” mean a macromolecule having the amino acid sequence of a native protein, that is a protein produced by a naturally-occurring and non-recombinant cell, or produced by a genetically-engineered or recombinant cell, and comprise molecules having the amino acid sequence of the native protein, or molecules having deletions from, additions to, and/or substitutions of one or more amino acids of the native sequence. The terms “polypeptide” and “protein” specifically encompass NAP proteins, or sequences that have deletions from, additions to, and/or substitutions of one or more amino acid of the NAP proteins NAP5, NAP6, rNAPc2, and rNAPc2/proline. The terms “polypeptide fragment” or “fragment” refer to a polypeptide that has an amino-terminal deletion, a carboxyl-terminal deletion, and/or an internal deletion as compared with the full-length native protein. Such fragments may also contain modified amino acids as compared with the native protein. In certain embodiments, fragments are about 5 to 500 amino acids long. For example, fragments may be at least 5, 6, 8, 10, 14, 20, 50, 70, 100, 110, 150, 200, 250, 300, 350, 400, 450 or 500 amino acids long.

The term “isolated protein” means that a subject protein (1) is free of at least some other proteins with which it would normally be found, (2) is essentially free of other proteins from the same source, e.g., from the same species, (3) is expressed by a cell from a different species, (4) has been separated from at least about 50% of polynucleotides, lipids, carbohydrates, or other materials with which it is associated in nature, (5) is operably associated (by covalent or noncovalent interaction) with a polypeptide with which it is not associated in nature, or (6) does not occur in nature. Genomic DNA, cDNA, mRNA or other RNA, of synthetic origin, or any combination thereof may encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide means an amino acid sequence wherein one or more amino acid residues are inserted into, deleted from and/or substituted into the amino acid sequence relative to another polypeptide sequence. Variants of the invention include fusion proteins.

A “derivative” of a polypeptide means a polypeptide that has been chemically modified in some manner distinct from insertion, deletion, or substitution variants, e.g., via conjugation to another chemical moiety.

It is understood that the polypeptides of the present invention may be modified, such that they are substantially identical to the NAP protein sequences, or fragments thereof. Polypeptide sequences are “substantially identical” when optimally aligned using such programs as GAP or BESTFIT using default gap weights, they share at least 80%, 90%, 95%, 96%, 97%, 98%, or 99% sequence identity. In certain embodiments, residue positions which are not identical differ by conservative amino acid substitutions. Conservative amino acid substitutions refer to the interchangeability of residues having similar side chains. For example, a group of amino acids having aliphatic side chains comprises glycine, alanine, valine, leucine, and isoleucine; a group of amino acids having aliphatic-hydroxyl side chains comprises serine and threonine; a group of amino acids having amide-containing side chain comprises asparagine and glutamine; a group of amino acids having aromatic side chains comprises phenylalanine, tyrosine, and tryptophan; a group of amino acids having basic side chains comprises lysine, arginine, and histadine; a group of amino acids having acidic side chains comprises aspartate and glutamate; and a group of amino acids having sulfur-containing side chains comprises cysteine and methionine. Preferred conservative amino acid substitution groups are: valine-leucine-isoleucine, phenylalanine-tyrosine, lysine-arginine, alanine-valine, aspartate-glutamate, and asparagine-glutamine.

As discussed herein, minor variations in the amino acid sequences of the NAP proteins of the invention are contemplated as being encompassed by the present invention, provided that the variations in the amino acid sequence maintain at least 75%, 80%, 90%, 95%, 96%, 97%, 98% or 99% sequence identity. Whether an amino acid change results in a functional peptide can readily be determined by assaying the specific activity of the polypeptide derivative. Assays are described in detail herein in Example 5.

Fragments or analogs of the NAP proteins of the invention can be readily prepared by those of ordinary skill in the art. Preferred amino- and carboxy-termini of fragments or analogs occur near boundaries of functional domains.

Structural and functional domains can be identified by comparison of the nucleotide and/or amino acid sequence data to public or proprietary sequence databases.

Computerized comparison methods are used to identify sequence motifs or predicted protein conformation domains that occur in other proteins of known structure and/or function. Methods to identify protein sequences that fold into a known three-dimensional structure are known in the art. Bowie et al., Science 253:164 (1991).

The term “effective amount” means an amount effective, at dosages and for periods of time necessary, to achieve the desired therapeutic or prophylactic result.

An “effective amount” of an anti-metastatic agent in reference to decreasing neoplastic cell growth, means an amount capable of decreasing, to some extent, the growth of some cancer or tumor cells. The term includes an amount capable of invoking a growth inhibitory, cytostatic and/or cytotoxic effect and/or apoptosis of the cancer or tumor cells.

A “therapeutically effective amount” in reference to the treatment of tumor, means an amount capable of invoking one or more of the following effects: (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but is not required to, result in the regression or rejection of the tumor, or (7) relief, to some extent, of one or more symptoms associated with the disorder. The therapeutically effective amount may vary according to factors such as the disease state, age, sex and weight of the individual and the ability of the anti-metastatic agent in combination with one or more anti-cancer agents to elicit a desired response in the individual. A “therapeutically effective amount” is also one in which any toxic or detrimental effects are outweighed by the therapeutically beneficial effects.

The phrases “treating cancer” and “treatment of cancer” mean to decrease, reduce, or inhibit the replication of cancer cells, decrease, reduce or inhibit the spread (formation of metastases) of cancer, decrease tumor size, decrease the number of tumors (i.e. reduce tumor burden), lessen or reduce the number of cancerous cells in the body, prevent recurrence of cancer after surgical removal or other anti-cancer therapies, or ameliorate or alleviate the symptoms of the disease caused by the cancer.

The term “anti-tumor activity” means a reduction in the rate of cell proliferation and hence a decline in growth rate of abnormal cells that arises during therapy. Anti-tumor activity also encompasses a reduction in tumor size. Such activity can be assessed using accepted animal models, such as the Namalwa and Daudi xenograft models of human B-cell lymphoma. See, e.g., Hudson et al., Leukemia 12:2029-2033 (1998) for a description of these animal models.

The term “tumor” means any neoplastic cell growth or proliferation, whether malignant or benign, whether in liquid or solid form and all pre-cancerous and cancerous cells and tissues.

The terms “cancer” and “cancerous” refer to or describe the physiological condition in mammals that is typically characterized by unregulated cell growth. A cancer refers to any of a number of conditions caused by the abnormal, uncontrolled growth of cells. Cells capable of causing cancer, called “cancer cells,” possess a number of characteristic properties such as uncontrolled proliferation, immortality, metastatic potential, rapid growth and proliferation rate, and certain typical morphological features. Often, cancer cells will be in the form of a tumor, but such cells may also exist alone or may be a non-tumorgenic cancer cell, such as a leukemia cell. A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer (e.g., CA125, PAP, PSA, CEA, AFP, HCG, CA 19-9, CA 15-3, CA 27-29, LDH, NSE, and others) and detecting a genotype indicative of a cancer (e.g., TP53, ATM, etc). However, a negative result in one or more of the above detection methods does not necessarily indicate the absence of cancer, e.g., a patient who has exhibited a complete response to a cancer treatment may still have a cancer, as evidenced by a subsequent relapse. Examples of cancer include, but are not limited to, carcinoma, lymphoma, blastoma, sarcoma, and leukemia.

Administration “in combination with” one or more further agents includes simultaneous (concurrent) or sequential administration in any order sufficient for both the anti-metastatic agent and the anti-cancer agent to act together for treating a proliferative disease such as cancer.

The term “additive” means the collective effect of separate components, such as an anti-metastatic agent and an anti-cancer agent, where the result of acting together is the sum of their individual effects.

The term “synergism” means the collective effect of separate components, such as an anti-metastatic agent and an anti-cancer agent, where the result of acting together is greater than the sum of the individual effects, i.e., greater than an additive effect.

The term “anti-cancer agent” means any agent that can be used to treat a cell proliferative disorder such as cancer, including cytotoxic agents, chemotherapeutic agents, radiotherapy and radiotherapeutic agents, targeted anti-cancer agents, and immunotherapeutic agents.

The term “anti-metastatic agent” means a substance that inhibits, reduces or decreases metastasis of cancer cells. Anti-metastatic agents include, substances that inhibit or reduce angiogenesis, tissue factor activity, factor VIIa activity, or tissue factor/factor VIIa complex activity. Examples include VEGF inhibitors, anti-VEGF antibodies (i.e. bevacizumab, AVASTIN®), non-anticoagulant heparins, low molecular weight heparins (LMWH, such as LOVENOX®), anti-tissue factor antibodies, CRA-5 (anti-fVIIa), BMS262084, TTP889, protein C, APC (Drotrecogin), sTM, Idraparinux, DX9065a, BAY59-7939, LY-51,7717, BMS-562247, DU-176b, otamixaban, razaxaban.

The term “cytotoxic agent” means a substance that decreases or inhibits the function of cells and/or causes destruction of cells, i.e., the substance is toxic to the cells. Typically, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, gelonin, mitegellin, restrictocin, phenomycin, neomycin, and the tricothecenes) or animals, e.g., cytotoxic RNases, such as extracellular pancreatic RNases; DNase I, including fragments and/or variants thereof.

A “chemotherapeutic agent” means a chemical compound that non-specifically decreases or inhibits the growth, proliferation, and/or survival of cancer cells. Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous. Such agents are often administered, and are often most effective, in combination, e.g., in the formulation CHOP.

Examples of chemotherapeutic agents contemplated by the present invention include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal and calicheamicin omega1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR), tegafur (UFTORAL), capecitabine (XELODA), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg., USA); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DFMO); retinoids such as retinoic acid; pharmaceutically acceptable salts, acids or derivatives of any of the above.

Also useful are combinations of two or more of the above such as CHOP (a combination of cyclophosphamide, doxorubicin, vincristine and prednisone) as well as the use of the constituents of CHOP either alone or in various combinations such as CO, CH, CP, COP, CHO, CHP, HO, HP, HOP, OP, etc.; CHOP and bleomycin (CHOP-BLEO); cyclophosphamide and fludarabine; cyclophosphamide, mitoxantrone, prednisone and vincristine; cyclophosphamide, dexamethasone, doxorubicin and vincristine (CAVD); CAV; cyclophosphamide, doxorubicin and prednisone; cyclophosphamide, mitoxantrone, prednisone and vincristine (CNOP); cyclophosphamide, methotrexate, leucovorin and cytarabine (COMLA); cyclophosphamide, dexamethasone, doxorubicin and prednisone; cylophosphamide, prednisone, procarbazine and vincristine (COPP); cylophosphamide, prednisone and vincristine (COP and CVP-1); cyclophosphamide and mitoxantrone; etoposide; mitoxantrone, ifosfamide and etoposide (MIV); cytarabine; methylprednisolone and cisplatin (ESHAP); methylprednisolone, cytarabine and cisplatin (ESAP); fludarabine, cytosine arabinoside (Ara-C) and G-CSF (FLAG); irinotecan, 5-FU (IFL); oxaliplatin, 5-FU, leucovorin (FOLFOX); oxaliplatin, irinotecan (IROX); leucovorin, 5-FU, irinotecan (FOLFIRI); methotrexate, leucovorin, doxorubicin, cyclophosphamide, vincristine, bleomycin and prednisone (MACOP-B); methotrexate, bleomycin, doxorubicin, cyclophosphamide, vincristine, and dexamethasone (m-BACOD); prednisone, cyclophosphamide, etoposide, cytarabine, bleomycin, vincristine, methotrexate and leucovorin (PROMACE-CYTABOM); etoposide, cyclophosphamide, vincristine, prednisone and bleomycin (VACOP-B); fludarabine and mitoxantrone; cisplatine, cytarabine and etoposide; desamethasone, fludarabine and mitoxantrone; chlorambucil and prednisone; busulfan and fludarabine; ICE; DVP; ATRA; Idarubicin, hoelzer chemotherapy regime; La La chemotherapy regime; ABVD; CEOP; 2-CdA; FLAG and IDA (with or without subsequent G-CSF treatment); VAD; M and P; C-Weekly; ABCM; MOPP; cisplatin, cytarabine and dexamethasone (DHAP), as well as the additional known chemotherapeutic regimens. Preparation and dosing schedules for such chemotherapeutic agents are also described in Chemotherapy Service Ed., M. C. Perry, Williams and Wilkins, Baltimore, Md. (1992).

Also included in this definition are anti-hormonal agents that act to regulate, reduce, block, or inhibit the effects of hormones that can promote the growth of cancer, and are often in the form of systemic or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR^{a}), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

The terms “radiation therapy” or “radiotherapeutic agents” mean the administration of radioactivity or radioactive compounds to a subject with cancer. Radiation decreases or inhibits the growth of dividing cells, such as cancer cells. Such therapy may include radiation from radioactive isotopes (e.g., phosphorous-32, copper-67, arsenic-77, rhodium-105, palladium-109, silver-111, tin-121, iodine-125 or 131, holmium-166, lutetium-177, rhenium-186 or 188, iridium-194, gold-199, astatium-21, yttrium-90, samarium-153, or bismuth-212). The radiation therapy may be whole body irradiation, or may be directed locally to a specific site or tissue in the body, such as the lung, colon, or prostate.

The term “targeted anti-cancer agents” means molecules directed to specific proteins, lipids, or other cellular components. Such targeted anti-cancer agents include monoclonal antibodies or other types of antibodies (i.e., fragments, single chain antibodies, bi-specific antibodies) or molecules (such as peptibodies) that target antigens. Examples of such immunotherapeutic targeted antibodies include without limitation bevacizumab (AVASTIN®, Genentech, South San Francisco, Calif., USA), tositumomab (BEXXAR®, GlaxoSmithKline, United Kingdom), alemtuzumab (CAMPATH®, Genzyme, Cambridge, Mass., USA), cetuximab (ERBITUX®, ImClone Systems Inc., New York, USA), trastuzumab (HERCEPTIN®, Genentech), gemtuzumab ozogamicin (MYLOTARG®, Wyeth, Madison, N.J., USA), rituximab (RITUXAN®, Biogen Idec, San Diego, Calif., USA), ibritumomab tiuxetan (ZEVALIN®, Biogen Idec), mitomomab (BEC2), C225, Oncolym, epratuzumab (Lymphocide), oregovomab (OVAREX®, ViRexx, Edmonton, Alberta, Canada), lintuzumab (SMART M195), apolizumab (SMART 1D10), VITAXIN® (Medimmune, Inc., Gaithersburg, Md., USA). Also captured by the term “targeted anti-cancer agents” are immunotoxins. By “immunotoxin” is meant an antibody- or antibody-like-toxin conjugate intended to destroy specific target cells (e.g., tumor cells) which bear antigens homologous to the antibody. Examples of toxins that are coupled to such antibodies include but are not limited to ricin A chain (RTA), blocked ricin (bIR), saporin (SAP), pokeweed antiviral protein (PAP) and Pseudomonas exotoxin (PE), and other toxic compounds, such as radioisotopes and other chemotherapeutic drugs, as described above.

The term “immunotherapeutic agent” means a compound that is an immunopotentiator or an immunosuppressant and is useful for treating diseases and disorders including cancer. Such agents include, without limitation, various cytokines and lymphokines, such as TGF-β, LAF, TCGF, BCGF, TRF, BAF, BDG, MP, LIF, OSM, TMF, PDGF, a number of interleukins, including IL-1α, IL-1β, IL-2, IL-3, IL-4, IL-5, IL-6, IL-7, IL-8, IL-9, IL-10, IL-11, IL-12, IL-13 and muteins of these molecules; interferons, such as but not limited to IFN-α, IFN-β, IFN-γ and muteins thereof; colony stimulating factors such as M-CSF, G-CSF, GM-CSF and muteins of GM-CSF; tumor necrosis factors, such as TNF-α and TNF-β and muteins of these molecules.

The terms “label” and “detectable label” refer to a molecule capable of detection, including, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used under the invention include, but are not limited to, horseradish peroxidase (HRP), fluorescein, FITC, rhodamine, dansyl, umbelliferone, dimethyl acridinium ester (DMAE), Texas red, luminol, NADPH and α- or β-galactosidase.

The term “subject” as used herein means a mammal, such as, but not limited to, domestic and farm animals and zoo, sports or pet animals, such as cow, monkey, horse, sheep, pig, cat, dog, mouse, rat, rabbit, guinea pig or human. Preferably the mammal is a human. A subject can be a human patient.

The term “biological sample” refers to a sample of tissue or fluid isolated from a subject such as, but not limited to, blood, plasma, platelets, serum, fecal matter, urine, bone marrow, bile, spinal fluid, lymph fluid, cerebrospinal fluid, samples of the skin, secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components. The samples detailed above need not necessarily be in the form obtained directly from the source. For example, the sample can be treated prior to use, such as, for example, by heating, centrifuging, etc. prior to analysis.

The phrase “pharmaceutically acceptable” vehicle, carrier or adjuvant means a non-toxic agent that can be tolerated by a recipient patient at the dosages and concentrations employed. Often the pharmaceutical carrier is an aqueous pH buffered solution. Representative non-limiting examples of such agents include human serum albumin, gelatin, ion exchangers, alumina, lecithin, buffer substances such as phosphates, citrate, glycine, antioxidants such as ascorbic acid, potassium sorbate and other organic acids, and salts or electrolytes such as protamine sulfate. Suitable vehicles are, for example, water, saline, phosphate-buffered saline, dextrose, glycerol, ethanol, hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI Americas, Inc., Bridgewater, N.J., USA), polyethylene glycol (PEG), and PLURONIC® (BASF, Florham Park, N.J., USA). Other suitable agents are well-known to those in the art. See, for example, Remington's Pharmaceutical Sciences, Mack Publishing Company, Easton, Pa., 19th edition, 1995. Actual methods of preparing such compositions are also known, or will be apparent, to those skilled in the art. See, e.g., Remington's Pharmaceutical Sciences, Mack Publishing Company, Easton, Pa., 19th edition, 1995.

The terms “Factor Xa” or “fXa” or “FXa” are synonymous and mean a serine protease within the blood coagulation cascade of enzymes that functions to form the enzyme thrombin as part of the prothrombinase complex.

The term “catalytically inactive fXa derivative” includes the zymogen Factor X (“fX” or “FX”), as well as other catalytically inactive fXa derivatives.

The phrase “inhibitory activity” means reducing or decreasing the activity of a molecule being acted upon.

The phrase “Factor Xa inhibitory activity” means an activity that inhibits, reduces or decreases the catalytic activity of fXa toward its substrate.

The phrase “Factor Xa selective inhibitory activity” means inhibitory activity that is selective toward Factor Xa compared to other related enzymes, such as other serine proteases.

The phrase “Factor Xa inhibitor” means a compound having Factor Xa inhibitory activity.

The terms “Factor VIIa/Tissue Factor” or “Tissue Factor/Factor VIIa” or “fVIIa/TF” or “FVIIa/TF” or “TF/fVIIa” are synonymous and mean a catalytically active complex of the serine protease coagulation factor VIIa (fVIIa) and the non-enzymatic protein Tissue Factor (TF), wherein the complex is assembled on the surface of a phospholipids membrane of defined composition.

The phrase “fVIIa/TF inhibitory activity” or “TF/fVIIa inhibitory activity” means an activity that inhibits, reduces or decreases the catalytic activity of the fVIIa/TF complex in the presence of fXa or catalytically inactive fXa derivatives (including the zymogen fX).

The phrase “fVIIa/TF selective inhibitory activity” means inhibitory activity that is selective toward fVIIa/TF compared to other related enzymes, such as other serine proteases, including fVIIa and fXa.

The phrase “fVIIa/TF inhibitor” means a compound having fVIIa/TF inhibitory activity in the presence of fXa or catalytically inactive fXa derivatives.

The phrase “serine protease” means an enzyme with a common three-dimensional fold, comprising a triad of the amino acids histadine, aspartic acid and serine that catalytically cleaves an amide bond, wherein the serine residue within the triad is involved in a covalent manner in the catalytic cleavage. Serine proteases are rendered catalytically inactive by covalent modification of the serine residue within the catalytic triad by diisopropylfluorophosphate (DFP).

The term “prothrombinase” means a catalytically active complex of the serine protease coagulation Factor Xa (fXa) and the non-enzymatic protein Factor Va (fVa), wherein the complex is assembled on the surface of a phospholipids membrane of defined composition.

The phrase “anticoagulant activity” means an activity that inhibits, reduces or decreases the clotting of blood, which includes the clotting of plasma.

The terms “selective,” “selectivity,” and permutations thereof, when referring to activity of a compound or composition toward a certain enzyme, mean that the compound or composition inhibits the specified enzyme with at least 10-fold higher potency than it inhibits other, related enzymes. Thus, the activity of the compound or composition is “selective” toward that specified enzyme.

The terms “NAP5” and “NAP6” mean isolated proteins of the NAP family. The preparation and sequence of NAP5 and NAP6 are described in U.S. Pat. No. 5,866,542.

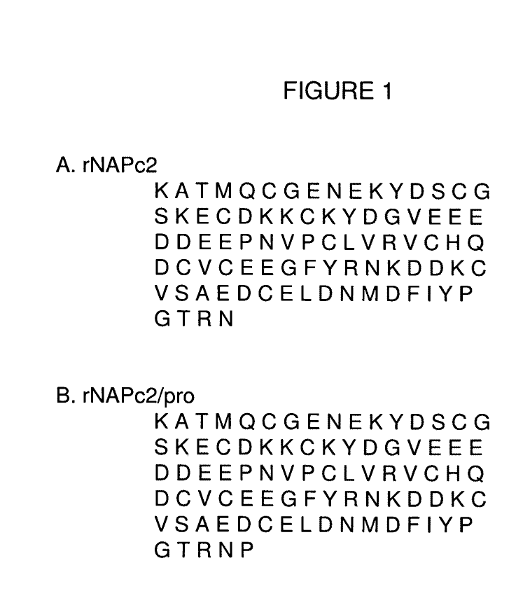

The term “rNAPc2” means a recombinant protein of the NAP family. The preparation and sequence of rNAPc2 is described in U.S. Pat. No. 5,866,542 and FIG. 1A herein (amino acid sequence of rNAPc2).

The terms “rNAPc2/proline,” “rNAPc2/pro” and “rNAPc2p” mean a recombinant protein having the amino acid sequence of rNAPc2 which has been modified to add a proline residue to the C-terminus of the sequence of rNAPc2 (see FIG. 1B herein).

Various aspects of the invention are described in further detail in the following subsections.

### II. NAP PROTEINS

Studies with various experimental metastatic tumor models have shown that, in order to metastasize, tumor cells need to migrate from the site of primary tumor into the bloodstream or lymphatic system, travel to a distant organ, arrest in the microvasculature, extravasate, and grow at the secondary site. In this multistep process, thrombin generation and fibrin formation appear to play important roles. Though not wishing to be bound by the following theory, it is believed that since a majority of the cancer cells that successfully enter the circulation are rapidly eliminated due to blood turbulence, in order to enhance survival, tumor cells tend to aggregate with each other or host cells such as platelets and lymphocytes. They form multicellular emboli (also called thrombi) that are often coated with fibrin. It is thought that these thrombi protect tumor cells against mechanical forces and stabilize the interaction of tumor cells with the microvasculature of the target organ. In addition, thrombin generation on the surface of the tumor cells assists in extravasation by inducing vasodilation of the endothelial cells of the microvasculature, degradation of endothelial cell matrix, and expression of cell-cell adhesion molecules such as intercellular adhesion molecule 1 (ICAM-1), P- and E-selectins on the surface of endothelial cells.

Tissue Factor (TF) is a 45 kD transmembrane glycoprotein that consists of an extracellular domain, a transmembrane region, and an intracellular domain (see Bach, CRC Crit. Rev. Biochem. 23:339 (1998)). TF binds and allosterically activates factor VII (fVIIa) and the complex TF/VIIa is responsible for thrombin generation via activation of factors IX and X and is the major initiator of blood clotting under physiological conditions. In addition, TF is overexpressed by many cancers, including colorectal, melanoma, and lung cancers and the level of TF positively correlates with the progression of malignancy. TF plays critical roles in intracellular signaling of metastasis and angiogenesis as well as thrombin generation and pathological thrombosis. TF expression is induced by a variety of proinflammatory cytokines, and although it is not synthesized by cells within the vasculature, such as monocytes and endothelial cells, following vascular injury expression is induced thereby activating the blood coagulation cascade. Accordingly, it is thought that inflammation, blood coagulation and blood vessel formation are interdependent processes and that such processes play an essential role in metastasis via the bloodstream.

The present invention provides methods for the treatment of diseases and processes that are mediated by, or associated with, abnormal or undesirable cellular proliferation. The compositions comprise isolated naturally occurring or synthetic molecules such as proteins, peptides or protein fragments containing all or an active fragment of an anti-metastatic agent administered in combination with one or more anti-cancer agents. In a preferred embodiment, the anti-metastatic agent is a NAP protein. NAP proteins are characterized by the presence of at least one NAP domain and demonstrate serine protease inhibition and/or anticoagulant activity. It is believed that the NAP domain is responsible for the observed anticoagulant activity of these proteins. Furthermore, rNAPc2 has been shown to be a potent inhibitor of angiogenesis (Hembrough et al., Cancer Res. 63:2997-3000 (2003)).

Certain NAP proteins of the present invention demonstrate specificity toward inhibiting a particular component in the coagulation cascade, such as fXa or the fVIIa/TF complex. The NAP proteins NAP5 and NAP6 each have one NAP domain and demonstrate selective inhibition of Factor Xa. rNAPc2 and rNAPc2/pro each have a single NAP domain and demonstrate selective inhibition of the fVIIa/TF complex when fXa, or a catalytically active or inactive derivative thereof, is present. According to one aspect of the present invention, these inhibitors in combination with other anti-cancer agents are used as a treatment for cancer by reducing or inhibiting undesirable cell proliferation thereby reducing tumor burden and metastasis.

A. NAP Proteins Having Factor Xa Inhibitory Activity

U.S. Pat. No. 5,866,542 describes a family of proteins termed “Nematode-Extracted Anticoagulant Proteins (NAP proteins), one class of which is described as having Factor Xa inhibitory activity. This class of proteins includes isolated proteins having Factor Xa inhibitory activity and having one or more NAP domains, wherein each NAP domain includes the sequence: Cys-A1-Cys-A2-Cys-A3-Cys-A4-Cys-A5-Cys-A6-Cys-A7-Cys-A8-Cys-A9-Cys-A10 (“Formula II”), wherein

(a) A1 is an amino acid sequence of 7 to 8 amino acid residues;

(b) A2 is an amino acid sequence;

(c) A3 is an amino acid sequence of 3 amino acid residues;

(d) A4 is an amino acid sequence;

(e) A5 is an amino acid sequence of 3 to 4 amino acid residues;

(f) A6 is an amino acid sequence;

(g) A7 is an amino acid residue;

(h) A8 is an amino acid sequence of 11 of 12 amino acid residues;

(i) A9 is an amino acid sequence of 5 to 7 amino acid residues;

(j) A10 is an amino acid sequence; wherein each of A2, A4, A6, and A10 has an independently selected number of independently selected amino acid residues and each sequence is selected such that each NAP domain has in total less than about 120 amino acid residues.

Suitable NAP proteins with this aspect of the invention have at least one NAP domain. Preferred are polypeptides having at least one NAP domain substantially the same as the NAP domains of NAP5 (SEQ ID NO: 1) and NAP6 (SEQ ID NO: 2). The NAP proteins NAP5 and NAP6 each have one NAP domain and are preferred NAP proteins according to this aspect of the invention. NAP5 is a 77-amino acid protein (SEQ ID NO: 1) and NAP6 is a 75-amino acid protein (SEQ ID NO: 2) originally isolated from the hookworm Ancylostoma caninum.

Preferred NAP proteins according to one embodiment of this aspect of the invention are those in which A2 is an amino acid sequence of 3 to 5 amino acid residues, A4 is an amino acid sequence of 6 to 19 amino acid residues, A6 is an amino acid sequence of 3 to 5 amino acid residues, and A10 is an amino acid sequence of 5 to 25 amino acid residues.

In another preferred embodiment of this aspect of the invention, A3 has the sequence Glu-A3ₐ-A3_{b}, wherein A3ₐ and A3_{b} are independently selected amino acid residues. More preferably, A3ₐ is selected from the group consisting of Ala, Arg, Pro, Lys, Ile, H is, Leu, and Thr, and A3_{b} is selected from the group consisting of Lys, Thr, and Arg.

In an additional preferred embodiment of this aspect of the invention, A4 is an amino acid sequence having a net anionic charge.

According to this aspect of the invention, a preferred A7 amino acid residue is Val or Ile.

Another preferred embodiment of this aspect of the invention is one in which A8 includes the amino acid sequence A8ₐ-A8_{b}-A8_{c}-A8_{d}-A8ₑ-A8_{f}-A8_{g} (SEQ ID NO: 5), wherein

(a) A8ₐ is the first amino acid residue in A8;

(b) at least one of A8ₐ and A8_{b} is selected from the group consisting of Glu or Asp; and

(c) A8_{c} through A8_{g} are independently selected amino acid residues.

Preferably, A8_{c} is Gly, A8_{d} is selected from the group consisting of Phe, Tyr, and Leu, A8ₑ is Tyr, A8_{f} is Arg, and A8_{g} is selected from Asp and Asn.

NAP proteins NAP5 and NAP6 include the amino acid sequence Glu-Ile-Ile-His-Val (SEQ ID NO: 6) in A10, and are preferred NAP proteins according to this embodiment of the invention.

In one embodiment, the present invention is directed to methods of using a suitable NAP protein having fXa inhibitory activity wherein

(a) A3 has the sequence Glu-A3ₐ-A3_{b}, wherein A3ₐ and A3_{b} are independently selected amino acid residues;

(b) A4 is an amino acid sequence having a net anionic charge;

(c) A7 is selected from the group consisting of Val and Ile;

(d) A8 includes an amino acid sequence selected from the group consisting of Gly-Phe-Tyr-Arg-Asp (SEQ ID NO: 7), Gly-Phe-Tyr-Arg-Asn (SEQ ID NO: 8), Gly-Tyr-Tyr-Arg-Asp (SEQ ID NO: 9), Gly-Tyr-Tyr-Arg-Asn (SEQ ID NO: 10), and Gly-Leu-Tyr-Arg-Asp (SEQ ID NO:11); and

(e) A10 includes an amino acid sequence selected from the group consisting of Glu-Ile-Ile-His-Val (SEQ ID NO: 6), Asp-Ile-Ile-Met-Val (SEQ ID NO: 12), Phe-Ile-Thr-Phe-Ala-Pro (SEQ ID NO: 13), and Met-Glu-Ile-Ile-Thr (SEQ ID NO: 14).

Use of pharmaceutical compositions comprising one or more of these NAP proteins having fXa inhibitory activity in combination with one or more anti-cancer agent and a pharmaceutically acceptable carrier are contemplated by this embodiment. NAP proteins used according to the methods of the present invention have at least one NAP domain. The NAP proteins NAP5 (SEQ ID NO: 1) and NAPE (SEQ ID NO: 2) each have one NAP domain and are preferred NAP proteins according to this aspect of the invention.

In another preferred embodiment, a suitable NAP protein for use in the methods of the present invention is one wherein

(a) A3 is selected from the group consisting of Glu-Ala-Lys, Glu-Arg-Lys, Glu-Pro-Lys, Glu-Lys-Lys, Glu-Ile-Thr, Glu-His-Arg, Glu-Leu-Lys, and Gly-Thr-Lys;

(b) A4 is an amino acid sequence having a net anionic charge;

(c) A7 is Val or Ile;

(d) A8 includes an amino acid sequence selected from the group consisting of A8ₐ-A8_{b}-Gly-Phe-Tyr-Arg-Asp (SEQ ID NO: 15), A8ₐ-A8_{b}-Gly-Phe-Tyr-Arg-Asn (SEQ ID NO: 16), A8ₐ-A8_{b}-Gly-Tyr-Tyr-Arg-Asp (SEQ ID NO: 17), A8ₐ-A8_{b}-Gly-Tyr-Tyr-Arg-Asn (SEQ ID NO: 18), and A8ₐ-A8_{b}-Gly-Leu-Tyr-Arg-Asp (SEQ ID NO: 19), wherein at least one of A8ₐ and A8_{b} is Glu or Asp;

(e) A9 is an amino acid sequence of five amino acid residues; and

(f) A10 includes an amino acid sequence selected from the group consisting of Glu-Ile-Ile-His-Val (SEQ ID NO: 6), Asp-Ile-Ile-Met-Val (SEQ ID NO: 12), Phe-Ile-Thr-Phe-Ala-Pro (SEQ ID NO: 13), and Met-Glu-Ile-Ile-Thr (SEQ ID NO: 14).

Use of pharmaceutical compositions comprising one or more of these NAP proteins having fXa inhibitory activity in combination with one or more anti-cancer agent and a pharmaceutically acceptable carrier are contemplated by this embodiment. NAP proteins used according to the methods of the present invention have at least one NAP domain. The NAP proteins NAP5 (SEQ ID NO: 1) and NAP6 (SEQ ID NO: 2) each have one NAP domain and are preferred NAP proteins according to this embodiment of the invention.

Certain preferred NAP proteins having fXa inhibitory activity as described above are derived from a nematode species. A preferred nematode species is selected from the group consisting of Ancylostoma caninum, Ancylostoma ceylanicum, Ancylostoma duodenale, Necator americanus, and Heligomosomoides polygyrus. These NAP proteins may conveniently be prepared by recombinant means. Particularly preferred are the NAP proteins NAP5 (SEQ ID NO: 1) and NAP6 (SEQ ID NO: 2) derived from Ancylostoma caninum.

B. NAP Proteins Having Factor VIIa/TF Inhibitory Activity

U.S. Pat. No. 5,866,542 describes a family of proteins termed “Nematode-Extracted Anticoagulant Proteins (NAP proteins), one class of which is described as having Factor VIIa/TF inhibitory activity. This class of proteins includes isolated proteins having Factor VIIa/TF inhibitory activity and having one or more NAP domains, wherein each NAP domain includes the sequence: Cys-A1-Cys-A2-Cys-A3-Cys-A4-Cys-A5-Cys-A6-Cys-A7-Cys-A8-Cys-A9-Cys-A10 (“Formula III”), wherein

(a) A1 is an amino acid sequence of 7 to 8 amino acid residues;

(b) A2 is an amino acid sequence;

(c) A3 is an amino acid sequence of 3 amino acid residues;

(d) A4 is an amino acid sequence;

(e) A5 is an amino acid sequence of 3 to 4 amino acid residues;

(f) A6 is an amino acid sequence;

(g) A7 is an amino acid residue;

(h) A8 is an amino acid sequence of 11 of 12 amino acid residues;

(i) A9 is an amino acid sequence of 5 to 7 amino acid residues;

(j) A10 is an amino acid sequence; wherein each of A2, A4, A6, and A10 has an independently selected number of independently selected amino acid residues and each sequence is selected such that each NAP domain has in total less than about 120 amino acid residues.

Suitable NAP proteins with this aspect of the invention have at least one NAP domain. Preferred are polypeptides having at least one NAP domain substantially the same as the NAP domain of rNAPc2 (SEQ ID NO: 3, FIG. 1A). The NAP protein rNAPc2 has one NAP domain and is a preferred NAP according to this aspect of the invention. Also preferred is rNAPc2/proline (SEQ ID NO: 4, FIG. 1B). rNAPc2 is a naturally occurring polypeptide that was originally isolated from the hookworm Ancylostoma caninum. The recombinant protein is a single chain, non-glycosylated 85 amino acid protein. The anticoagulant activity of rNAPc2 results from its ability to inhibit the TF/fVIIa complex which is responsible for initiation of the process leading to blood clot formation. By inhibiting this complex, rNAPc2 blocks PAR-2 activation and prevents thrombin generation. rNAPc2/proline is a recombinant protein that has the sequence of rNAPc2 with an additional proline residue at the C-terminus (SEQ ID NO: 4).

Preferred NAP proteins include those in which A2 is an amino acid sequence of 3 to 5 amino acid residues, A4 is an amino acid sequence of 6 to 19 amino acid residues, A6 is an amino acid sequence of 3 to 5 amino acid residues, and A10 is an amino acid sequence of 5 to 25 amino acid residues.

In certain preferred NAP proteins, A3 has the sequence Asp-A3ₐ-A3_{b}, wherein A3ₐ and A3_{b} are independently selected amino acid residues. More preferably, A3 is Asp-Lys-Lys.

In certain preferred NAP proteins, A4 is an amino acid sequence having a net anionic charge.

Also preferred are NAP proteins in which A5 has the sequence A5ₐ-A5_{b}-A5_{c}-A5_{d} (SEQ ID NO: 20), wherein A5ₐ through A5_{d} are independently selected amino acid residues. Preferably, A5ₐ is Leu and A5 is Arg.

In certain preferred NAP proteins, a preferred A7 amino acid residue is Val or Ile, more preferably Val.

Certain preferred NAP proteins include those in which A8 includes the amino acid sequence A8ₐ-A8_{b}-A8_{c}-A8_{d}-A8ₑ-A8_{f}-A8_{g} (SEQ ID NO: 5), wherein

(a) A8ₐ is the first amino acid residue in A8;

(b) at least one of A8ₐ and A8_{b} is selected from the group consisting of Glu or Asp; and

(c) A8_{c} through A8_{g} are independently selected amino acid residues.

Preferably, A8_{c} is Gly, A8_{d} is selected from the group consisting of Phe, Tyr, and Leu, A8ₑ is Tyr, A8_{f} is Arg, and A8_{g} is selected from Asp and Asn. A preferred A8_{c}-A8_{d}-A8ₑ-A8_{f}-A8_{g} sequence is Gly-Phe-Tyr-Arg-Asn (SEQ ID NO: 8).

In one embodiment, the present invention is directed to methods of using a suitable NAP protein having factor VIIa/TF inhibitory activity wherein

(a) A3 has the sequences Asp-A3ₐ-A3_{b}, wherein A3ₐ and A3_{b} are independently selected amino acid residues;

(b) A4 is an amino acid sequence having a net anionic charge;

(c) A5 has the sequence A5ₐ-A5_{b}-A5_{c}-A5_{d} (SEQ ID NO: 20), wherein A5ₐ through A5_{d} are independently selected amino acid residues; and

(d) A7 is selected from the group consisting of Val and Ile.

Use of pharmaceutical compositions comprising one or more of these NAP proteins having factor VIIa/TF inhibitory activity in combination with one or more anti-cancer agent and a pharmaceutically acceptable carrier are contemplated by this embodiment. NAP proteins used according to the methods of the present invention have at least one NAP domain. The NAP proteins rNAPc2 and rNAPc2/proline each have one NAP domain and are preferred NAP proteins according to this embodiment of the invention.

In another preferred embodiment, a suitable NAP protein for use in the methods of the present invention is one wherein

(a) A3 is Asp-Lys-Lys

(b) A4 is an amino acid sequence having a net anionic charge;

(c) A5 has the sequence A5ₐ-A5_{b}-A5_{c}-A5_{d} (SEQ ID NO: 20), wherein A5ₐ through A5_{d} are independently selected amino acid residues;

(d) A7 is Val;

(e) A8 includes an amino acid sequence A8ₐ-A8_{b}-Gly-Phe-Tyr-Arg-Asn (SEQ ID NO: 16), wherein at least one of A8ₐ and A8_{b} is Glu or Asp.

Use of pharmaceutical compositions comprising such NAP proteins in combination with one or more anti-cancer agent and a pharmaceutically acceptable carrier are also contemplated by this invention. These NAP proteins have at least one NAP domain. The NAP proteins rNAPc2 (SEQ ID NO: 3) and rNAPc2/proline (SEQ ID NO: 4) each have one NAP domain and are preferred NAP proteins according to this embodiment of the invention.

Certain preferred NAP proteins having Factor VIIa/TF inhibitory activity as described above are derived from a nematode species. A preferred nematode species is selected from the group consisting of Ancylostoma caninum, Ancylostoma ceylanicum, Ancylostoma duodenale, Necator americanus, and Heligomosomoides polygyrus. These NAP proteins may conveniently be prepared by recombinant means. Particularly preferred are the NAP proteins rNAPc2 and rNAPc2/proline. rNAPc2 was derived from Ancylostoma caninum using recombinant methods.

U.S. Pat. No. 5,866,542 describes the preparation of recombinant NAP proteins, including rNAPc2 and rNAPc2/proline.

### III. COMBINATION THERAPY

The present invention relates to methods for decreasing or inhibiting tumor growth or tumor metastasis, comprising administering to a subject in need thereof an effective amount of an anti-metastatic agent together with one or more anti-cancer agents. The anti-cancer agent can be administered prior to, concurrently with, overlapping with, or subsequent to administration of the anti-metastatic agent. In addition, the anti-metastatic agent and anti-cancer agent may be administered as one composition or as separate compositions.

Combination therapies contemplated by the invention may be useful in decreasing or inhibiting unwanted endothelial cell proliferation, decreasing the occurrence of cancer, decreasing or preventing the recurrence of cancer, or decreasing or preventing the spread or metastasis of cancer. A combination therapy of an anti-metastatic agent and one or more anti-cancer agent may be used to diminish established cancer e.g., decrease the number of cancer cells present and/or decrease tumor burden, or ameliorate at least one manifestation or side effect of cancer. As such, combination therapy refers to the administration of an anti-metastatic agent, preferably a NAP protein, most preferably rNAPc2/proline, and one or more anti-cancer agent which includes, but is not limited to, cytotoxic agents, chemotherapeutic agents, targeted anti-cancer agents, cancer vaccines, anti-angiogenic agents, cytokines, hormone therapies, radiation therapy and anti-metastatic agents.

According to the methods of the present invention, there is no requirement for the combined results to be additive of the effects observed when each treatment (i.e., anti-metastatic agent and anti-cancer agent) is conducted separately. Although at least additive effects are generally desirable, any increased anti-tumor effect above one of the single therapies is beneficial. Furthermore, the invention does not require the combined treatment to exhibit synergistic effects.

To practice the combination therapy according to the invention, an anti-metastatic agent in combination with one or more anti-cancer agent may be administered to a subject in need thereof in a manner effective to result in anti-cancer activity within the subject. The anti-metastatic agent and anti-cancer agent are provided in amounts effective and for periods of time effective to result in their combined presence and their combined actions in the tumor environment as desired. To achieve this goal, the anti-metastatic agent and anti-cancer agent may be administered to the subject simultaneously, either in a single composition, or as two or more distinct compositions using different administration routes.

Alternatively, the anti-metastatic agent may precede, or follow, the anti-cancer agent treatment by, e.g., intervals ranging from minutes to weeks. In certain embodiments wherein the anti-cancer agent and the anti-metastatic agent are applied separately to the subject, the time period between the time of each delivery is such that the anti-cancer agent and anti-metastatic agent are able to exert a combined effect on the tumor. In a particular embodiment, it is contemplated that both the anti-cancer agent and the anti-metastatic agent are administered within about 5 minutes to about two weeks of each other.

In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the anti-metastatic agent and the anti-cancer agent. The anti-metastatic agent and one or more anti-cancer agent (the combination therapy) may be administered once, twice or at least the period of time until the condition is treated, palliated or cured. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months, once every six months or may be administered continuously via a minipump. The combination therapy may be administered via an oral, mucosal, buccal, intranasal, inhalable, intravenous, subcutaneous, intramuscular, parenteral, intratumor or topical route. The combination therapy may be administered at a site distant from the site of the tumor. The combination therapy generally will be administered for as long as the tumor is present provided that the combination therapy causes the tumor or cancer to stop growing or to decrease in weight or volume.

In one embodiment, an anti-metastatic agent is administered, in combination with one or more anti-cancer agents, for a short treatment cycle to a cancer patient to treat cancer. The duration of treatment with the anti-cancer agent may vary according to the particular anti-cancer agent used. The invention also contemplates discontinuous administration or daily doses divided into several partial administrations. An appropriate treatment time for a particular anti-cancer agent will be appreciated by the skilled artisan, and the invention contemplates the continued assessment of optimal treatment schedules for each anti-cancer agent.

In a particular embodiment, the anti-metastatic agent is administered to the subject twice-weekly via subcutaneous injection. Subsequently, the anti-cancer agent is administered about two weeks after the initial administration of anti-metastatic agent. It is contemplated that each cycle of combination therapy takes about two weeks. The duration of the combination therapy may take 1, 2, 3, 4 or more cycles. In a preferred embodiment, the anti-metastatic agent is rNAPc2/pro and the anticancer agent is the chemotherapeutic combination FOLFOX or FOLFIRI. In a particular embodiment, the anti-cancer targeted anti-cancer agent bevacizumab (AVASTIN®) is administered in addition to FOLFOX or FOLFIRI.

The present invention contemplates at least one cycle, preferably more than one cycle during which the combination therapy is administered. An appropriate period of time for one cycle will be appreciated by the skilled artisan, as will the total number of cycles, and the interval between cycles. The invention contemplates the continued assessment of optimal treatment schedules for each anti-metastatic agent and anti-cancer agent.

The invention also provides for more than one administration of either the anti-metastatic agent or the anti-cancer agent. The anti-metastatic agent and anti-cancer agent may be administered interchangeably, on alternate days or weeks; or a sequence of anti-metastatic treatment may be given, followed by one or more treatments of anti-cancer agent therapy.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, FOLFIRI consists of intravenous leucovorin (200 mg/m²) as a 2 hour infusion and irinotecan (180 mg/m²) as a 90 min infusion followed by bolus 5-FU (400 mg/m²) and a 46 hour infusion of 5-FU (2400 mg/m²) repeated every two weeks. FOLFOX consists of intravenous leucovorin (200 mg/m²) as a 2 hour infusion and oxaliplatin (85 mg/m²) on day 1 as a 2 hour infusion concurrent with 5-FU, followed by bolus 5-FU (400 mg/m²) and a 46 hour infusion of 5-FU (2400 mg/m²) repeated every two weeks. Additional treatment with bevacizumab (AVASTIN®) consists of intravenous administration of 5 mg/kg bevacizumab over 90 min for the initial dose, and if well-tolerated, bevacizumab can be given over 60 min for the next dose and over 30 min as a maintenance dose and is repeated every two weeks.

The present invention also provides for the combination of an anti-metastatic agent with radiotherapy (i.e., any mechanism for inducing DNA damage locally within tumor cells such as γ-irradiation, X-rays, UV-irradiation, microwaves, electronic emissions and the like). Combination therapy using the directed delivery of radioisotopes to tumor cells is also contemplated, and may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

The present invention also provides for combination therapy using immunotherapeutic agents. Various cytokines may be employed in such combined approaches with an anti-metastatic agent. Cytokines are administered according to standard regimens, consistent with clinical indications such as the condition of the patient and relative toxicity of the cytokine. Uteroglobins may also be used to prevent or inhibit metastases (U.S. Pat. No. 5,696,092, incorporated herein by reference).

In yet another embodiment, an anti-metastatic agent is administered in combination with a cancer vaccine. Examples of cancer vaccines include, but are not limited to, autologous cells or tissues, non-autologous cells or tissues, carcinoembryonic antigen, alpha-fetoprotein, human chorionic gonadotropin, BCG live vaccine, melanocyte lineage proteins (e.g., gp100, MART-1/MelanA, TRP-1 (gp75), tyrosinase, widely shared tumor-specific antigens (e.g., BAGE, GAGE-1, GAGE-2, MAGE-1, MAGE-3, N-acetylglucosaminyltransferase-V, p15), mutated antigens that are tumor-specific (e.g., β-catenin, MUM-1, CDK4), nonmelanoma antigens (e.g., HER-2/neu (breast and ovarian carcinoma), human papillomavirus-E6, E7 (cervical carcinoma), MUC-1 (breast, ovarian and pancreatic carcinoma)). Human tumor antigens can be recognized by lymphocytes, such as T cells (See, e.g., Robbins and Kawakami, Curr. Opin. Immunol. 8:628-36 (1996)). Cancer vaccines need not be purified preparations.

In yet another embodiment, an anti-metastatic agent is administered in association with a hormonal treatment.

In another embodiment, an anti-metastatic agent is administered in combination with one or more anti-angiogenic agents.

The anti-metastatic agent may be conjugated to radionuclides using indirect labeling where a chelating agent is covalently attached to a protein and at least one radionuclide is inserted into the chelating agent. See, for example, the chelating agents and radionuclides described in Srivastava and Mease, Int. J. Rad. Appl. Instrum. B. 18:589-603 (1991). Alternatively, the anti-metastatic agent may be labeled using direct labeling where a label, such as a radionuclide is covalently attached directly to a protein (typically via an amino acid residue). For example, the peptide may be biosynthesized or may be synthesized by chemical amino acid synthesis using amino acid precursors involving, for example, fluorine-19 in place of hydrogen. Labels such as technetium-99m or iodine-123, rhenium-186, rhenium-188, and indium-111 can be attached via a cysteine residue in the peptide. Yttrium-90 can be attached via a lysine residue. The iodogen method (Franker et al., Biochem. Biophys. Res. Commun. 80:49-57 (1978)) can be used to incorporate iodine-123.

Conjugates of an anti-metastatic agent and a cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987).

Further, the invention provides an embodiment wherein an anti-metastatic agent is linked to an enzyme that converts a prodrug into a cytotoxic drug. The enzymes cleave the non-toxic prodrug into the toxic drug, which leads to tumor cell death. Suitable prodrug enzymes include thymidine kinase (TK), xanthine-guanine phosphoribosyltransferase (GPT) gene from E. coli or E. coli cytosine deaminase (CD), or hypoxanthine phosphoribosyl transferase (HPRT). Additional representative examples of enzymes and associated prodrug molecules include alkaline phosphatase and various toxic phosphorylated compounds such as phenolmustard phosphate, doxorubicin phosphate, mitomycin phosphate and etoposide phosphate; β-galactosidase and N-[4-(β-D-galactopyranosyl) benzyloxycarbonyl]-daunorubicin; azoreductase and azobenzene mustards; β-glucosidase and amygdalin; β-glucuronidase and phenolmustard-glucuronide and epirubicin-glucuronide; carboxypeptidase A and methotrexate-alanine; cytochrome P450 and cyclophosphamide or ifosfamide; DT diaphorase and 5-(aziridine-1-yl)-2,4,dinitrobenzamide (CB1954) (Cobb et al., Biochem. Pharmacol 18:1519 (1969), Knox et al., Cancer Metastasis Rev. 12:195 (1993)); β-glutamyl transferase and β-glutamyl p-phenylenediamine mustard; nitroreductase and CB1954 or derivatives of 4-nitrobenzyloxycarbonyl; glucose oxidase and glucose; xanthine oxidase and hypoxanthine; and plasmin and peptidyl-p-phenylenediamine-mustard.

Conjugates of an anti-metastatic agent and one or more small molecule toxins, such as calcheamicin, maytansinoids, a trichothecene, and CC1065, and the derivatives of these toxins that have toxin activity, are also contemplated herein.

### VII. PHARMACEUTICAL COMPOSITIONS

### A. Exemplary Formulations

In certain embodiments, the invention also provides compositions comprising an anti-metastatic agent and one or more anti-cancer agent together with one or more of the following: a pharmaceutically acceptable diluent; a carrier; a solubilizer; an emulsifier; a preservative; and/or an adjuvant. Such compositions may contain an effective amount of an anti-metastatic agent and/or an anti-cancer agent. Thus, the use of an anti-metastatic agent and one or more anti-cancer agents that are provided herein in the preparation of a pharmaceutical composition of a medicament is also included. Such compositions can be used in the treatment of a variety of diseases such as cancer.

The anti-metastatic agents may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular disease or disorder targeted. The compositions also preferably include pharmaceutically acceptable vehicles, carriers or adjuvants, well known in the art.

Acceptable formulation components for pharmaceutical preparations are nontoxic to recipients at the dosages and concentrations employed. In addition to the anti-metastatic agents and anti-cancer agents that are provided, compositions according to the invention may contain components for modifying, maintaining or preserving, for example, the pH, osmolarity, viscosity, clarity, color, isotonicity, odor, sterility, stability, rate of dissolution or release, adsorption or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18^{th} Ed., (A. R. Gennaro, ed.), 1990, Mack Publishing Company), hereby incorporated by reference.

The primary vehicle or carrier in a pharmaceutical composition may be either aqueous or non-aqueous in nature. Suitable vehicles or carriers for such compositions include water for injection, physiological saline solution or artificial cerebrospinal fluid, possibly supplemented with other materials common in compositions for parenteral administration. Neutral buffered saline or saline mixed with serum albumin are further exemplary vehicles. Compositions comprising an anti-metastatic agent and one or more anti-cancer agent may be prepared for storage by mixing the selected composition having the desired degree of purity with optional formulation agents in the form of a lyophilized cake or an aqueous solution. Further the anti-metastatic agent and anti-cancer agent may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration typically is sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

Additional pharmaceutical methods may be employed to control the duration of action of an anti-metastatic agent in a therapeutic application. Control release preparations can be prepared through the use of polymers to complex or adsorb the polypeptide. For example, biocompatible polymers include matrices of poly(ethylene-co-vinyl acetate) and matrices of a polyanhydride copolymer of a stearic acid dimer and sebacic acid. Sherwood et al., Bio/Technology 10:1446 (1992). The rate of release of a polypeptide from such a matrix depends upon the molecular weight of the protein, the amount of polypeptide within the matrix, and the size of dispersed particles. Saltzman et al., Biophys. J. 55:163 (1989); Sherwood et al., supra. Other solid dosage forms are described in Remington's Pharmaceutical Sciences, Mack Publishing Company, Easton, Pa., 19th edition, 1995.

The above compositions can be administered using conventional modes of delivery including, but not limited to, intravenous, intraperitoneal, oral, intralymphatic, subcutaneous administration, intraarterial, intramuscular, intrapleural, intrathecal, and by perfusion through a regional catheter. Local administration to a tumor in question is also contemplated by the present invention. Eye drops can be used for intraocular administration. When administering the compositions by injection, the administration may be by continuous infusion or by single or multiple boluses. For parenteral administration, the anti-metastatic agents may be administered in a pyrogen-free, parenterally acceptable aqueous solution comprising the desired anti-metastatic agents and anti-cancer agents in a pharmaceutically acceptable vehicle. A particularly suitable vehicle for parenteral injection is sterile distilled water in which an anti-metastatic agent and one or more anti-cancer agent are formulated as a sterile, isotonic solution, properly preserved.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

In particular embodiments, the anti-metastatic agent may be formulated as either a frozen liquid composition or a lyophilized composition. In addition to the anti-metastatic agent, the frozen liquid and lyophilized compositions may also include a bulking/osmolarity agent. Preferably, mannitol is incorporated in a concentration of about 2% to about 10% weight by volume (w/v).

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

A preferred liquid composition to be frozen will contain in addition to the solubilized NAP protein, sodium phosphate in a concentration from about 50 mM to about 100 mM and sodium chloride in a concentration from about 50 mM to about 100 mM, at a pH of about 7.0.

In a particular embodiment, the frozen liquid composition contains a NAP protein, preferably rNAPc2/pro, in a concentration of about 1 mg/ml, sodium phosphate in a concentration of about 65 mM and sodium chloride in a concentration of about 80 mM, at a pH of about 7.0.

For all compositions in accordance with this invention, the NAP protein is present in a concentration of about 0.5 mg/ml to about 2 mg/ml, preferably with a concentration of about 1 mg/ml.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

In certain embodiments, anti-metastatic agents may be administered ex vivo. In such instances, cells, tissues or organs that have been removed from the patient are exposed to or cultured with an anti-metastatic agent. The cultured cells may then be implanted back into the patient or a different patient or used for other purposes.

In certain embodiments, anti-metastatic agents can be delivered by implanting certain cells that have been genetically engineered into a subject, using methods such as those described herein, to express and secrete the polypeptide. Such cells may be animal or human cells, and may be autologous, heterologous, or xenogenic, or may be immortalized. In order to decrease the chance of an immunological response, the cells may be encapsulated to avoid infiltration of surrounding tissues. Encapsulation materials are typically biocompatible, semi-permeable polymeric enclosures or membranes that allow the release of the protein product(s) but prevent the destruction of the cells by the patient's immune system or by other detrimental factors from the surrounding tissues.

### B. Dosage

Typically, administration of an anti-metastatic agent or pharmaceutical composition comprising an anti-metastatic agent is commenced at lower dosage levels, with dosage levels being increased until the desired therapeutic effect is achieved which would define a therapeutically effective amount. For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

### VIII. THERAPEUTIC USES

The present invention provides methods for treating cancer (i.e. decrease or reduce undesired endothelial cell proliferation or decrease or reduce metastasis) by administering an anti-metastatic agent in combination with one or more anti-cancer agent.

Tumors treatable by the present invention also include solid phase tumors/malignancies (i.e., carcinomas) locally advanced tumors and human soft tissue sarcomas. Carcinomas include those malignant neoplasms derived from epithelial cells that infiltrate (invade) the surrounding tissues and give rise to metastastic cancers, including lymphatic metastases. Adenocarcinomas are carcinomas derived from glandular tissue, or which form recognizable glandular structures. Another broad category of cancers includes sarcomas, which are tumors whose cells are embedded in a fibrillar or homogeneous substance like embryonic connective tissue. The invention also enables treatment of cancers of the myeloid or lymphoid systems, including leukemias, lymphomas and other cancers that typically do not present as a tumor mass, but are distributed in the vascular or lymphoreticular systems.

The type of cancer or tumor cells that may be amenable to treatment according to the invention include, for example, B-cell leukemias, T-cell leukemias, B-cell lymphomas, T-cell lymphomas, myelomas, Hodgkin's disease, and non-Hodgkin's lymphoma, gastrointestinal cancers including esophageal cancer, stomach cancer, colon cancer, colorectal cancer, polyps associated with colorectal neoplasms, pancreatic cancer and gallbladder cancer, cancer of the adrenal cortex, ACTH-producing tumor, bladder cancer, brain cancer including intrinsic brain tumors, neuroblastomas, astrocytic brain tumors, gliomas, and metastatic tumor cell invasion of the central nervous system, Ewing's sarcoma, head and neck cancer including mouth cancer and larynx cancer, kidney cancer including renal cell carcinoma, liver cancer, lung cancer including small and non-small cell lung cancers, malignant peritoneal effusion, malignant pleural effusion, skin cancers including malignant melanoma, tumor progression of human skin keratinocytes, squamous cell carcinoma, basal cell carcinoma, and hemangiopericytoma, mesothelioma, Kaposi's sarcoma, bone cancer including osteomas and sarcomas such as fibrosarcoma and osteosarcoma, cancers of the female reproductive tract including uterine cancer, endometrial cancer, ovarian cancer, ovarian (germ cell) cancer and solid tumors in the ovarian follicle, vaginal cancer, cancer of the vulva, and cervical cancer; breast cancer (small cell and ductal), penile cancer, prostate cancer, retinoblastoma, testicular cancer, thyroid cancer, trophoblastic neoplasms, and Wilms' tumor.

The invention is particularly illustrated herein in reference to treatment of certain types of experimentally defined cancers. In these illustrative treatments, standard state-of-the-art in vitro and in vivo models have been used. These methods can be used to identify agents that can be expected to be efficacious in in vivo treatment regimens. However, it will be understood that the method of the invention is not limited to the treatment of these tumor types, but extends to any cancer derived from any organ system. As demonstrated in Example 1, combination therapy of rNAPc2/pro and 5-FU or AVASTIN® reduced tumor size in a murine human colorectal cancer model.

The present invention also provides for a preventative or prophylactic treatment of subjects who present with benign or precancerous tumors. It is not believed that any particular type of tumor should be excluded from treatment using the present invention. However, the type of tumor cells may be relevant to the use of the invention in combination with secondary therapeutic agents, particularly chemotherapeutic agents and targeted anti-cancer agents.

### IX. KITS

The present invention provides kits for producing single-dose or multi-dose administration units of an anti-metastatic agent and one or more anti-cancer agent. The kit comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). Such kits will generally contain in a suitable container a pharmaceutically acceptable formulation of an anti-metastatic agent and one or more anti-cancer agent. The kits may also contain other pharmaceutically acceptable formulations, either for diagnosis or combined therapy. For example, such kits may contain any one or more of a range of chemotherapeutic or radiotherapeutic drugs; anti-angiogenic agents; anti-metastatic agents; targeted anti-cancer agents; cytotoxic agents; and/or other anti-cancer agents.

The kits may have a single container that contains the anti-metastatic agent and one or more anti-cancer agent, with or without additional components, or they may have distinct containers for each desired agent. Where combined therapeutics are provided, a single solution may be pre-mixed, either in a molar equivalent combination, or with one component in excess of the other. Alternatively, each of the anti-metastatic agents and other anti-cancer agent components of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

The kits may also contain a means by which to administer the anti-metastatic agent and anti-cancer agent to an animal or patient, e.g., one or more needles or syringes, or even an eye dropper, pipette, or other such like apparatus, from which the formulation may be injected into the animal or applied to a diseased area of the body. The kits of the present invention will also typically include a means for containing the vials, or such like, and other component in close confinement for commercial sale, such as, e.g., injection or blow-molded plastic containers into which the desired vials and other apparatus are placed and retained.

The label or package insert indicates that the composition is used for treating cancer, for example colorectal cancer.

The invention having been described, the following examples are offered only by way of illustration and not limitation. The present invention may be better understood by reference to the following non-limiting Examples, which are provided only as exemplary of the invention. The following examples are presented to more fully illustrate the preferred embodiments of the invention. They should in no way be construed, however, as limiting the broader scope of the invention.

### Example 1

### Efficacy of rNAPc2 In Combination with 5-FU and/or AVASTIN® Using a Human Colorectal Cancer Murine Model

To determine the effect of rNAPc2/pro in combination with 5-fluorouracil (5-FU) in colorectal cancer, a murine xenograft colorectal tumor model was used. Human colorectal cancer cells (HCT116) were cultured in DMEM medium supplemented with 10% fetal bovine serum (FBS), harvested at log growth phase, enumerated and resuspended in HBSS buffer. Eight to ten week old female nude mice (NU/NU) were inoculated subcutaneously with 1×10⁷ cells/mouse in a volume of 100 μl. Tumors were allowed to develop to an average size of 50 to 100 mm³ before the treatment started (defined as day 0). HCT116 tumor xenograft-bearing mice were randomized into various treatment conditions and the treatment regimens lasted for 14 days.

The mice were treated as follows (6 mice/group):

1) Vehicle control;

2)ᵣNAPc2/pro (300 μg/kg, daily IP injection for 14 days);

3) LOVENOX® (1.5 mg/kg, daily IP injection for 14 days);

4) 5-FU (10 mg/kg, every-other-day IP injection for 14 days);

5) AVASTIN® (0.5 mg/kg, twice-weekly IP injection for 14 days);

6)rNAPc2/pro+5-FU;

7) LOVENOX®+5-FU;

8) AVASTIN®+5-FU;

9)rNAPc2/pro+AVASTIN®;

10) LOVENOX®+AVASTIN®; and

11)rNAPc2+5-FU+AVASTIN®.

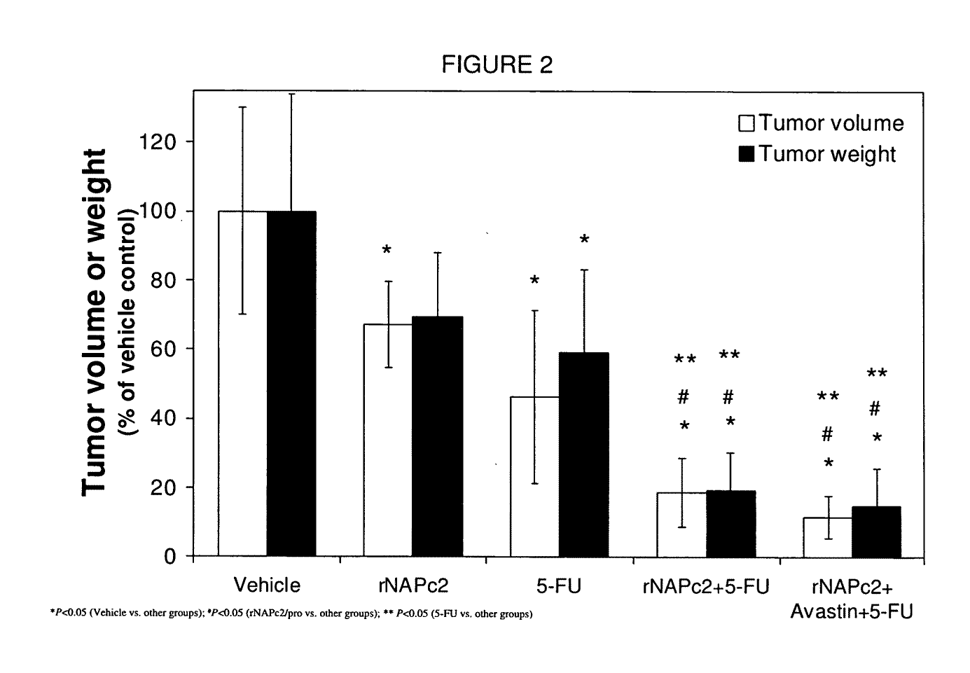

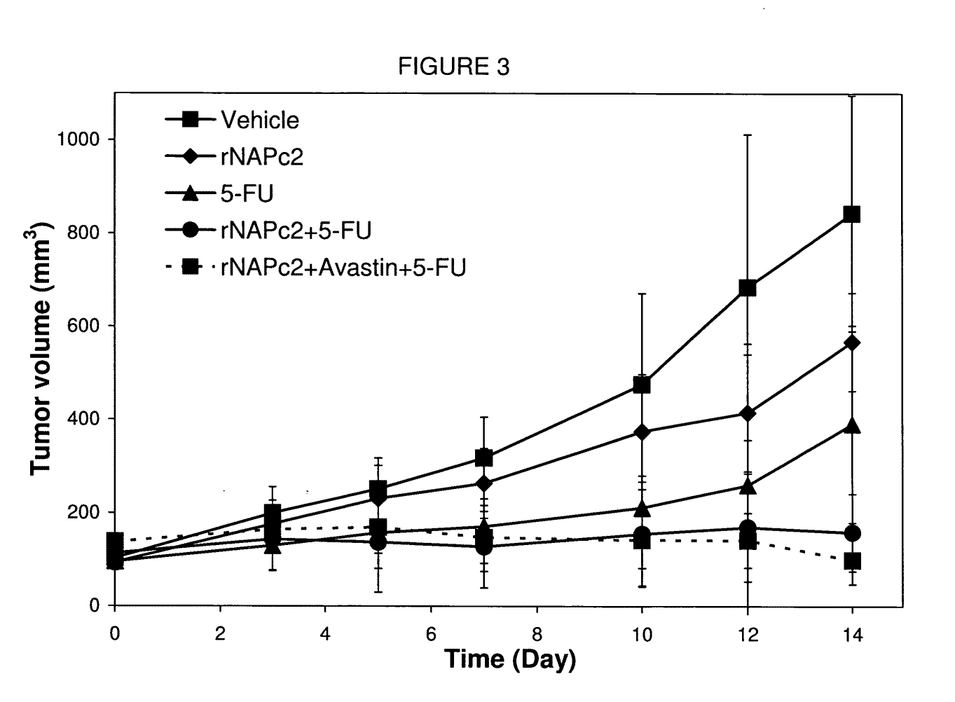

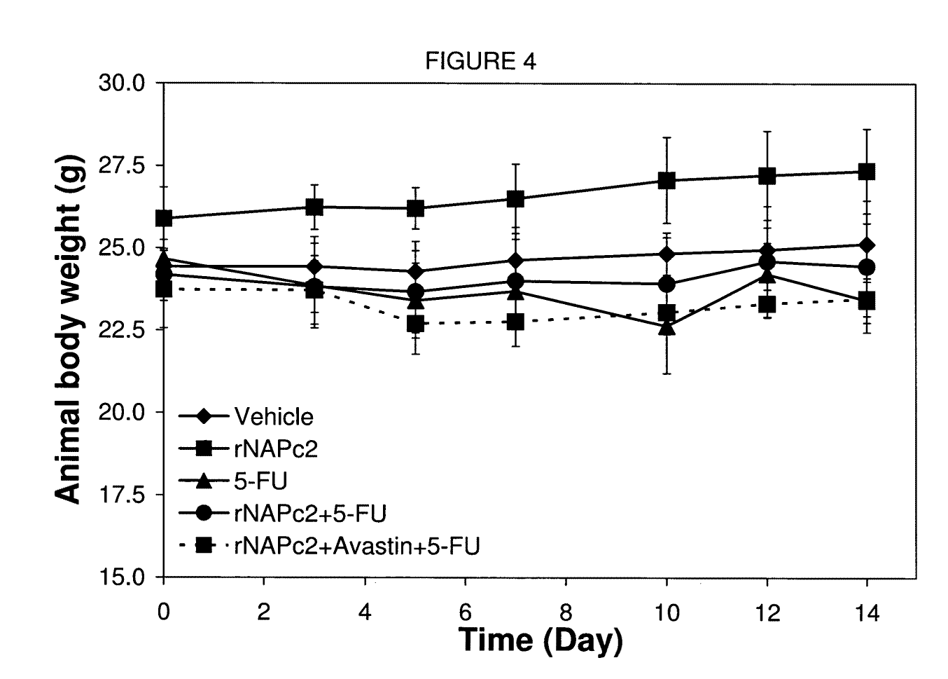

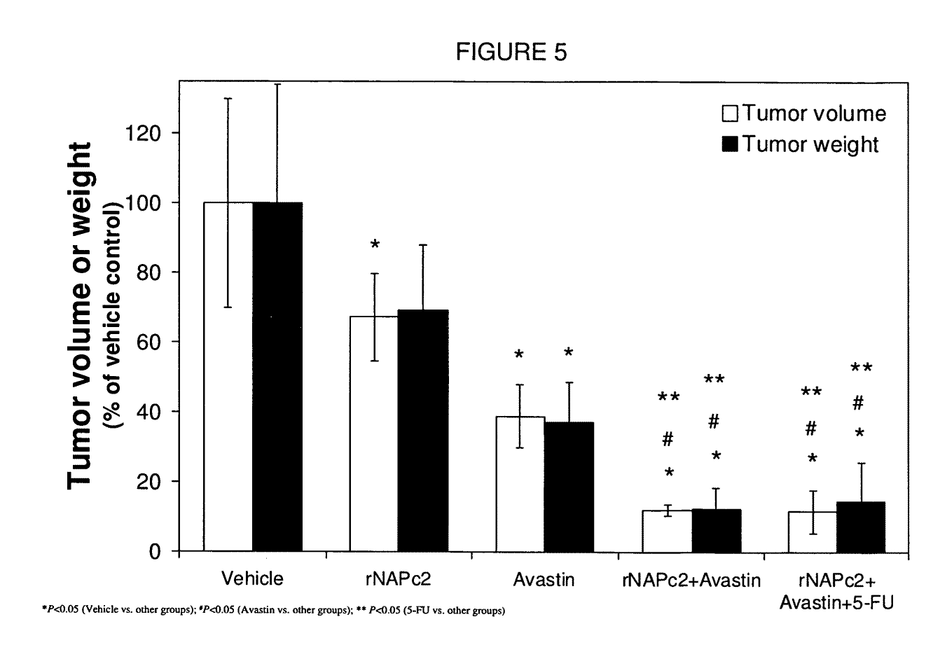

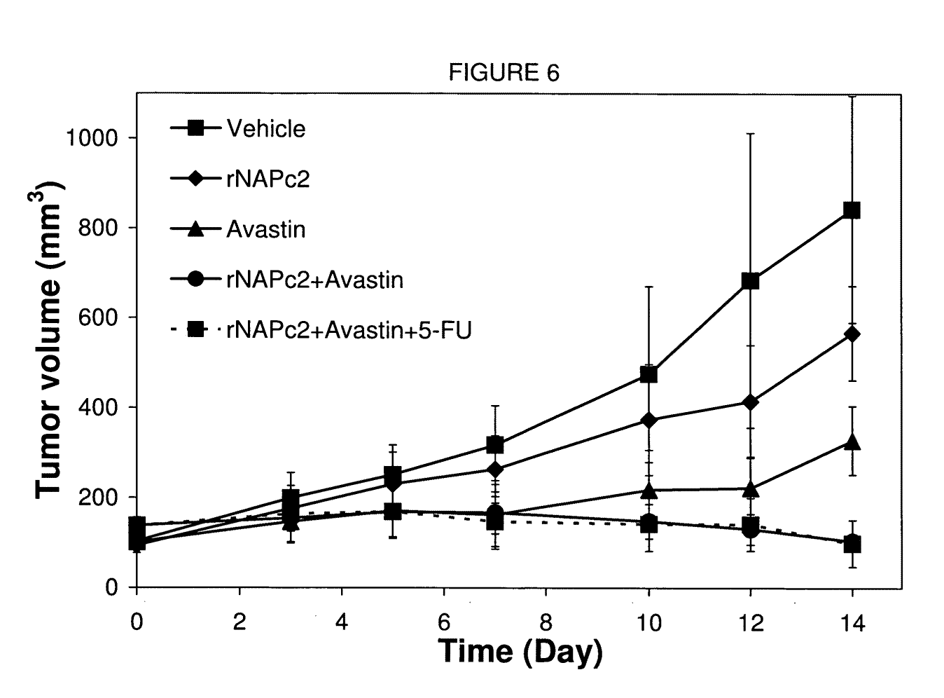

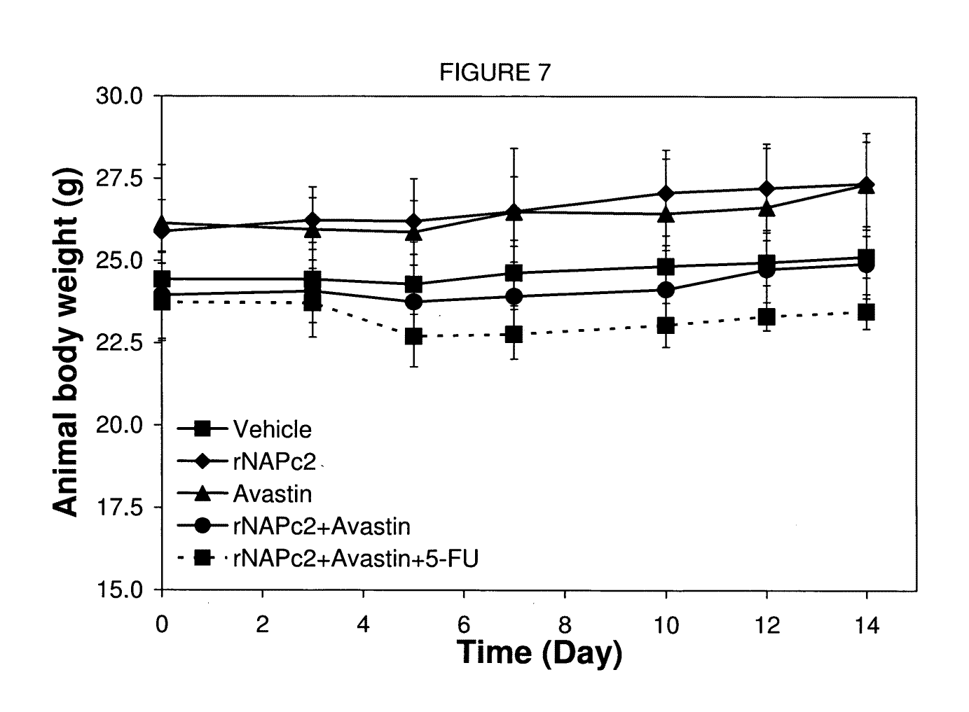

The mice were monitored every-other-day for body weight, signs of morbidity (i.e., hemorrhage) and mortality. On day 14, mice were extirpated for HCT116 tumor xenografts and the tumors were weighed and measured. Tumor size was calculated as follows: tumor volume (size)=(length)×(width²)/2 (mm³). Compared to vehicle control, rNAPc2/pro treatment alone reduced tumor weight by 31%, 5-FU treatment alone reduced tumor weight by 41% (P<0.05), and AVASTIN® treatment alone reduced tumor weight by 63% (P<0.05). The combined treatment of rNAPc2/pro and 5-FU further reduced tumor weight by 81% (P<0.05) (see FIGS. 2 and 5). A timecourse of the above-mentioned treatments are displayed in FIGS. 3 and 6. The combined treatment of rNAPc2/pro and AVASTIN® reduced tumor weight by 87% (P<0.05). The doses and dosing regimens of rNAPc2/pro used in the murine model of colorectal cancer were well tolerated by the recipient mice without major complications of hemorrhage or other adverse effects (see FIGS. 4 and 7 for effect of the treatments on animal body weight). The results of the combined administration of rNAPc2/pro and 5-FU or AVASTIN® suggest that rNAPc2/pro in combination with an anti-cancer agent may be an effective method of treating human colorectal cancer patients.

### Example 2

### Murine Model of Pulmonary Metastasis of Colorectal Cancer

To determine the effect of rNAPc2/pro in treating pulmonary tumor metastasis, a murine colorectal cancer-pulmonary metastasis mouse model was used. Murine colorectal cancer cells (CT26) were cultured in RPMI 1640 medium supplemented with 10% FBS, harvested at log growth phase, enumerated and resuspended in HBSS buffer. Nine to eleven week old female syngenic BALB/c mice were inoculated via tail vein injection with 1×10⁶ cells/mouse in a volume of 100 μl (defined as day 0). CT26-injected mice were randomized into various treatment conditions and the treatment regimens lasted for 11 days.

The mice were treated as follows (8 mice/group):

1) Naïve control;

2) CT26/saline control;

3) CT26/rNAPc2/pro (10 μg/kg/day);

4) CT26/rNAPc2/pro (30 μg/kg/day);

5) CT26/rNAPc2/pro (100 μg/kg/day);

6) CT26/rNAPc2/pro (300 μg/kg/day);

7) CT26/rNAPc2/pro (1 mg/kg/day);

8) CT26/rNAPc2/pro (2.5 mg/kg/day); and

9) CT26/LOVENOX® (1.5 mg/kg/day, IP injection).

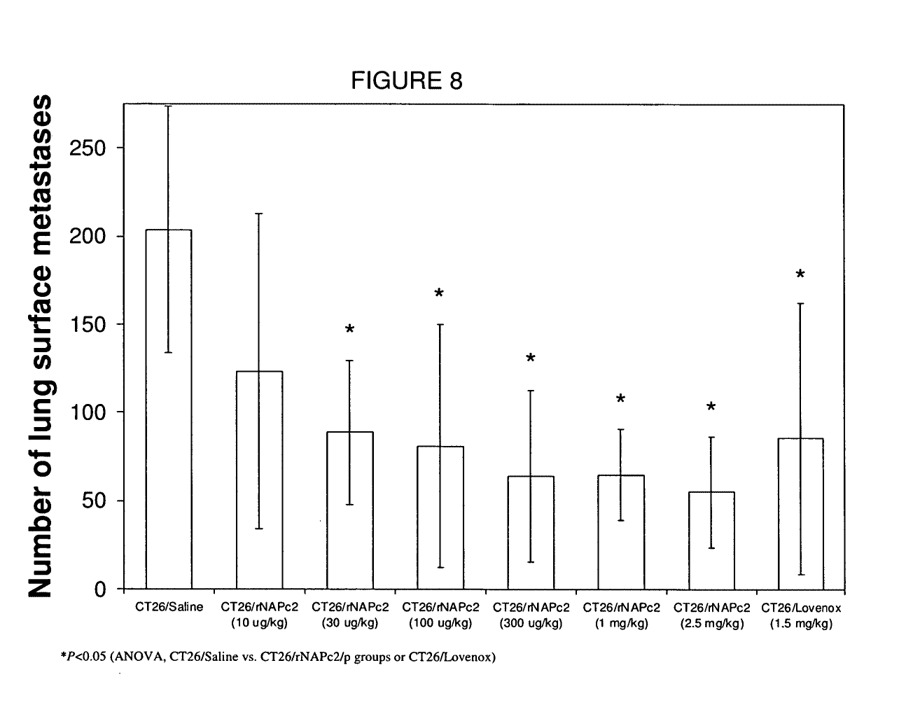

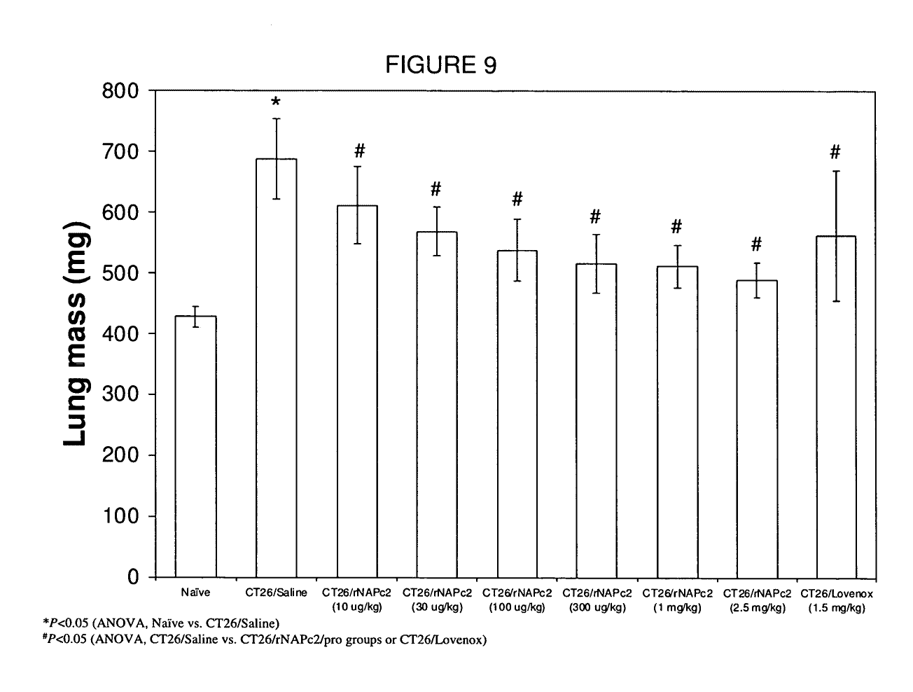

The mice were monitored every-other-day for body weight, signs of morbidity (i.e., hemorrhage) and mortality. On day 11, the lungs were surgically removed, weighed and the number of lung surface metastases were counted in a blinded fashion. The other internal organs were examined for signs of hemorrhage. Administration of rNAPc2/pro reduced the number of lung surface metastases (FIG. 8) and reduced lung mass (FIG. 9) when compared to saline control mice in a dose-dependent manner. In some of the mice, no lung surface metastases were observed (Table 1). rNAPc2/pro was well-tolerated up to 2.5 mg/kg/day in mice with no hemorrhage-related mortality. However, some local hemorrhage was noticed in internal organs in some of the mice treated with 2.5 mg/kg rNAPc2/pro and 1.5 mg/kg LOVENOX®. No significant difference in animal body weight was observed between all experimental groups. The results suggest that administration of rNAPc2/pro may be an effective method of reducing metastasis in human colorectal cancer patients.

TABLE 1

| | Number of mice with |
|---|---|
| | no observable lung |
| Treatment Group | surface metastases |
| CT26/saline | 0 |
| CT26/rNAPc2/pro (10 μg/kg/day) | 1 |
| CT26/rNAPc2/pro (30 μg/kg/day) | 0 |
| CT26/rNAPc2/pro (100 μg/kg/day) | 1 |
| CT26/rNAPc2/pro (300 μg/kg/day) | 1 |
| CT26/rNAPc2/pro (1 mg/kg/day) | 0 |
| CT26/rNAPc2/pro (2.5 mg/kg/day) | 1 |
| CT/26/LOVENOX ® (1.5 mg/kg/day) | 2 |

To determine if the effect is TF-dependent, this experiment may also be performed using CT26 cells in which endogenous TF is downregulated using short hairpin RNA (shRNA). Murine colorectal cancer cells (CT26) transformed with TF shRNA are cultured in RPMI 1640 medium supplemented with 10% FBS, harvested at log growth phase, enumerated and resuspended in HBSS buffer. Nine to eleven week old female syngenic BALB/c mice are inoculated via tail vein injection with 1×10⁶ cells/mouse in a volume of 100 μl (defined as day 0). CT26-injected mice are randomized into various treatment conditions and the treatment regimens lasts for 11 days.

The mice can be treated as follows (8 mice/group):

1) CT26 parental/saline;

2) CT26 parental/rNAPc2/pro (10 μg/kg/day);

3) CT26 parental/rNAPc2/pro (100 μg/kg/day);

4) CT26 with TF shRNA/saline;

5) CT26 with TF shRNA/rNAPc2/pro (10 μg/kg/day); and

6) CT26 with TF shRNA/rNAPc2/pro (100 μg/kg/day).

The mice are monitored every other day for body weight, signs of morbidity (i.e., hemorrhage) and mortality. On day 11, the lungs are surgically removed, weighed and the number of lung surface metastases are counted in a blinded fashion. The other internal organs are examined for signs of hemorrhage.

### Example 3

### Murine Model of Hepatic Metastasis of Colorectal Cancer

To determine the effect of rNAPc2/pro in treating hepatic tumor metastasis, a murine colorectal cancer-hepatic metastasis mouse model was used. Human colorectal cancer cells (HCT116) were cultured in DMEM medium supplemented with 10% FBS, harvested at log growth phase, enumerated and resuspended in HBSS buffer. Eight to ten week old female nude mice (NU/NU) were inoculated via portal vein injection with 2×10⁶ HCT116 cells/mouse in a volume of 100 μl (defined as day 0). HCT116-injected mice were randomized into various treatment conditions and the treatment regimens lasted for 28 days.

The mice were treated as follows (8 mice/group):

1) Naïve control;

2) HCT116/vehicle control;

3) HCT116/rNAPc2/pro (300 μg/kg, daily IP injection for 28 days);

4) HCT116/CPT-11 (100 mg/kg, once-weekly IP injection for 4 weeks); and

5) HCT116/rNAPc2/pro+CPT-11.

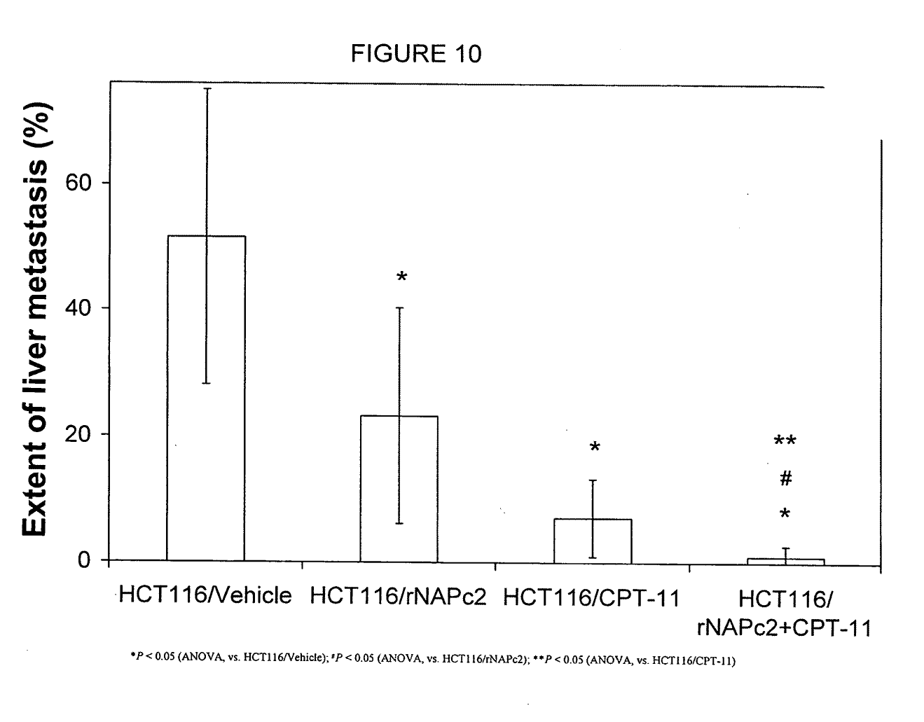

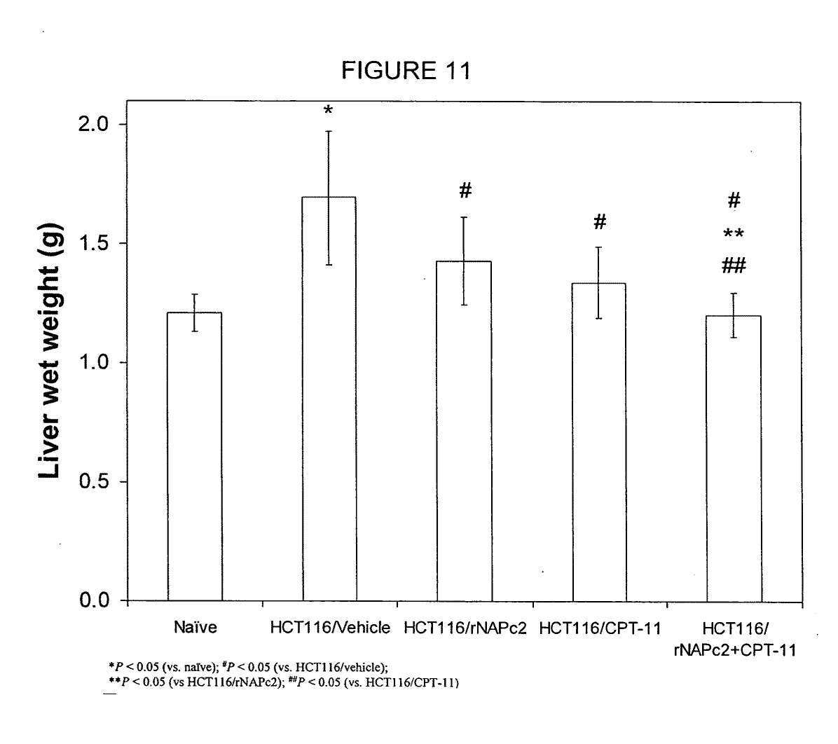

The mice were monitored every-other-day for body weight, signs of morbidity (i.e. hemorrhage) and mortality. On day 42, the livers were surgically removed, weighed and the number of liver surface metastases were counted in a blinded fashion. The other internal organs were examined for signs of hemorrhage. Administration of rNAPc2/pro reduced the number of liver surface metastases (FIG. 10) and reduced liver mass (FIG. 11) when compared to vehicle control mice. Furthermore, rNAPc2/pro had a synergistic effect with CPT-11 in inhibiting hepatic metastases. In some of the mice, no hepatic metastases were observed (Table 2).

TABLE 2

| Treatment Group | Hepatic metastasis-free events |
|---|---|
| HCT116/vehicle | 0% |
| HCT116/rNAPc2/pro | 0% |
| HCT116/CPT-11 | 0% |
| HCT116/rNAPc2/pro + CPT-11 | 50% |

No significant difference in animal body weight was observed between all experimental groups. The results of the combined administration of rNAPc2/pro+CPT-11 suggest that rNAPc2/pro in combination with an anti-cancer agent may be an effective method of reducing metastasis in human colorectal cancer patients.

### Example 4

### Murine Model of Spontaneous Intestinal Tumorigenesis Progression

To determine the effect of rNAPc2/pro in treating intestinal adenomas and adenocarcinomas, a murine spontaneous intestinal neoplasia mouse model was used: the Min mouse (The Jackson Laboratory, Bar Harbor, Me., USA). The Min mutation is an autosomal dominant heterozygous nonsense mutation of the mouse APC gene converting codon 850 from a leucine (TTG) to amber (TAG) (Su et al, Science 256:668 (1992)). It is homologous to the APC mutations carried in the germline of humans with familial adenomatous polyposis or that occur somatically in the majority of sporadic colon neoplasms (Jacoby et al, Cancer Res. 56:710 (1996)). The Min model is useful for testing anti-cancer agents targeted against early stage lesions because scores of adenomas, primarily in the small intestine, grow to a grossly detectable size in only one to three months on a defined genetic background (Moser et al, Science 247:322 (1990)).

Four to five week old Apc^{Min/+} or syngenic wild-type control (age- and sex-matched) were randomized into various treatment conditions and the treatment regimens lasted for 10 to 11 weeks.

The mice were treated as follows:

1) Wild-type/vehicle control (3 mice);

2) Wild-type/rNAPc2/pro (100 μg/kg, daily IP injection) (3 mice);

3) Apc^{Min/+}/vehicle control (6 mice);

4) Apc^{Min/+}/rNAPc2/pro (6 mice); and

5) Apc^{Min/+}/LOVENOX® (500 μg/kg, daily IP injection) (6 mice).

The mice were monitored every-other-day for body weight, signs of morbidity (i.e. hemorrhage) and mortality. On week 16, the intestines were removed, separated into proximal, middle and distal small intestine and colon, formed into “Swiss rolls” and processed for analysis. Mice were injected with 4 mg/mouse BrdU 2 h prior to harvest. Long-term administration of rNAPc2 suppressed formation of intestinal adenomas and adenocarcinomas in Apc^{Min/+} mice. The dosing regimens of rNAPc2 used in these studies were well-tolerated up to a three month period by recipient mice without major hemorrhage or other adverse effects. These results suggest that rNAPc2/pro may be an effective anti-tumor/metastatic agent to potentiate other anti-cancer therapies.

### Example 5

### Assays to Determine NAP Protein Activity

Any of these assays can be used to analyze the activity of NAP proteins, variants and fragments thereof and are described in detail in U.S. Pat. No. 6,090,916, herein incorporated by reference for all purposes.

A. Factor Xa Amidolytic Assay

The ability of NAP proteins to act as inhibitors of fXa catalytic activity is assessed by determining the NAP protein-induced inhibition of amidolytic activity catalyzed by the human fXa, as represented by Kᵢ* values.

The buffer used for all assays is HBSA (10 mM HEPES, pH 7.5, 150 mM sodium chloride, 0.1% bovine serum albumin).

The assay is conducted by combining in appropriate wells of a Corning microtiter plate, 50 μl of HBSA, 50 μl of the NAP protein diluted (0.025-25 nM) in HBSA (or HBSA alone for uninhibited velocity measurement), and 50 μl of the fXa enzyme diluted in HBSA (prepared from purified human fX) according to the method described by Bock, et al., Archives of Biochem. Biophys. 273:375 (1989), herein incorporated by reference. The enzyme is diluted into HBSA prior to the assay in which the final concentration was 0.5 nM. Following a 30 minute incubation at ambient temperature, 50 μl of the substrate S2765 (N-alpha-benzyloxycarbonyl-D-argininyl-L-glycyl-L-arginine-p-nitroanilide dihydrochloride), made up in deionized water followed by dilution in HBSA prior to the assay, are added to the wells yielding a final total volume of 200 μl and a final concentration of 250 μM (about 5×Kₘ). The initial velocity of chromogenic substrate hydrolysis is measured by the change in absorbance at 405 nm using a ThermoMax® Kinetic Microplate Reader (Molecular Devices, Sunnyvale, Calif., USA) over a 5 minute period in which less than 5% of the added substrate is utilized.

Ratios of inhibited pre-equilibrium, steady-state velocities containing NAP protein (Vᵢ) to the uninhibited velocity of free fXa alone (Vₒ) are plotted against the corresponding concentrations of NAP protein. These data are then directly fit to an equation for tight-binding inhibitors (Morrison and Walsh, Adv. Enzymol. 61:201-300 (1988) herein incorporated by reference), from which the apparent equilibrium dissociation inhibitory constant Kᵢ* is calculated.

B. Prothrombin Time (PT) and Activated Partial Thromboplastin Time (aPTT) Assays

The ex vivo anticoagulant effects of NAP proteins in human plasma are evaluated by measuring the prolongation of the activated partial thromboplastin time (aPTT) and prothrombin time (PT) over a broad concentration range of each inhibitor.

Respective measurements of aPTT and PT are made using the Coag-A-Mate RA4 automated coagulometer (General Diagnostics, Organon Technica, Oklahoma City, Okla., USA) using the Automated aPTT PLATELIN® L reagent (Organon Technica, Durham, N.C., USA) and SIMPLASTIN® Excel (Organon Technica) respectively, as initiators of clotting according to the manufacturer's instructions.

The assays are conducted by making a series of dilutions of each tested NAP protein in rapidly thawed plasma followed by adding 200 μl or 100 μl of the above referenced reagents to the wells of the assay carousel for the aPTT or PT measurements, respectively. Alternatively, the NAP proteins are serially diluted into HBSA and 10 μl of each dilution are added to 100 μl of normal human plasma in the wells of the Coag-A-Mate assay carousel, followed by addition of reagent.

Concentrations of NAP proteins are plotted against clotting time, and a doubling time concentration is calculated, i.e., a specified concentration of NAP protein that doubled the control clotting time of either the PT or the aPTT.

C. Prothrombinase Inhibition Assay

The ability of the NAP proteins to act as inhibitors of the activation of prothrombin by fXa that has been assembled into a physiologic prothrombinase complex is assessed by determining the respective inhibition constant, Kᵢ*.

Prothrombinase activity is measured using a coupled amidolytic assay, where a preformed complex of human fXa, human Factor Va (fVa), and phospholipid vesicles first activates human prothrombin to thrombin. The amidolytic activity of the generated thrombin is measured simultaneously using a chromogenic substrate. The chromogenic substrate Pefachrome t-PA (CH₃SO₂-D-hexahydrotyrosine-glycyl-L-arginine-p-nitroanilide) is reconstituted in deionized water prior to use. Phospholipid vesicles are made, consisting of phosphotidyl choline (67%, w/v), phosphatidyl glycerol (16%, w/v), phosphatidyl ethanolamine (10%, w/v), and phosphatidyl serine (7%, w/v) in the presence of detergent, as described by Ruf et al. (Methods in Enzymology 222: 209-224 (1993) herein incorporated by reference).

The prothrombinase complex is formed in a polypropylene test tube by combining fVa, fXa, and phospholipid vesicles (PLV) in HBSA containing 3 mM CaCl₂ for 10 min. In appropriate wells of a microtiter plate, 50 μl of the complex are combined with 50 μl of NAP protein diluted in HBSA, or HBSA alone (for Vₒ (uninhibited velocity) measurement). Following an incubation of 30 min at room temperature, the reactions are initiated by the addition of a substrate solution, containing human prothrombin and the chromogenic substrate for thrombin, Pefachrome tPA. The final concentration of reactants in a total volume of 150 μl of HBSA is: NAP (0.025-25 nM), fXa (250 fM), PLV (5 μM), prothrombin (250 nM), Pefachrome tPA (250 μM, 5×Kₘ), and CaCl₂ (3 mM).

The prothrombinase activity of fXa is measured as an increase in the absorbance at 405 nm over 10 min (velocity), exactly as described in Example 5A, under steady-state conditions. The absorbance increase is sigmoidal over time, reflecting the coupled reactions of the activation of prothrombin by the fXa-containing prothrombinase complex, and the subsequent hydrolysis of Pefachrome tPA by the generated thrombin. The data from each well are fit by reiterative, linear least squares regression analysis, as a function of absorbance versus time², as described (Carson, Comput. Prog. Biomed. 19: 151-157 (1985)) to determine the initial velocity (Vᵢ) of prothrombin activation. Ratios of inhibited steady-state initial velocities containing NAP (Vᵢ) to the uninhibited velocity of prothrombinase fXa alone (Vₒ) are plotted against the corresponding concentrations of NAP. These data are directly fit to the equation for tight-binding inhibitors, as in Example 5A above, and the apparent equilibrium dissociation inhibitory constant Kᵢ* is calculated.

D. In vitro Enzyme Assays for Activity Specificity Determination

The ability of NAP proteins to act as selective inhibitors of fXa catalytic activity or TF/fVIIa activity is assessed by determining whether the NAP proteins inhibit other enzymes in an assay at a concentration that is 100-fold higher than the concentration of the following related serine proteases: thrombin, Factor Xa, Factor Xla, Factor Xlla, kallikrein, activated protein C, plasmin, recombinant tissue plasminogen activator (rt-PA), urokinase, chymotrypsin, and trypsin. These assays also are used to determine the specificity of NAP proteins having serine protease inhibitory activity.

General Protocol For Enzyme Inhibition Assays

The buffer used for all assays is HBSA (Example 5A). All substrates are reconstituted in deionized water, followed by dilution into HBSA prior to the assay. The amidolytic assay for determining the specificity of inhibition of serine proteases is conducted by combining in appropriate wells of a microtiter plate, 50 μl of HBSA, 50 μl of NAP protein at a specified concentration diluted in HBSA, or HBSA alone (uninhibited control velocity, Vₒ), and 50 μl of a specified enzyme (see specific enzymes below). Following a 30 minute incubation at ambient temperature, 50 μl of substrate are added to triplicate wells. The final concentration of reactants in a total volume of 200 μl of HBSA is: NAP (75 nM), enzyme (750 μM), and chromogenic substrate (as indicated below). The initial velocity of chromogenic substrate hydrolysis is measured as a change in absorbance at 405 nm over a 5 minute period, in which less than 5% of the added substrate is hydrolyzed. The velocities of test samples, containing NAP (Vᵢ) are then expressed as a percent of the uninhibited control velocity (Vₒ) by the following formula: VᵢVₒ×100, for each of the enzymes.

Specific Enzyme Assays

(a) Thrombin Assay

Thrombin catalytic activity is determined using the chromogenic substrate Pefachrome t-PA (CH₃SO₂-D-hexahydrotyrosine-glycyl-L-arginine-p-nitroaniline) at a final concentration of 250 μM (about 5×Kₘ).

(b) Factor Xa Assay

Factor Xa catalytic activity is determined using the chromogenic substrate S-2765 (N-benzyloxycarbonyl-D-arginine-L-glycine-L-arginine-p-nitroaniline) at a final concentration of 250 μM (about 5×Kₘ). Purified human fXa is activated and prepared from fX as described (Bock, et al. Arch. Biochem. Biophys. 273:375-388 (1989)).

(c) Factor XIa Assay

Factor XIa catalytic activity is determined using the chromogenic substrate S-2366 (L-Pyroglutamyl-L-prolyl-L-arginine-p-nitroaniline) at a final concentration of 750 μM.

(d) Factor XIIa Assay

Factor XIIa catalytic activity is determined using the chromogenic substrate Spectrozyme FXIIa (H-D-CHT-L-glycyl-L-arginine-p-nitroaniline) at a final concentration of 100 μM.

### (e) Kallikrein Assay

Kallikrein catalytic activity is determined using the chromogenic substrate S-2302 (H-D-prolyl-L-phenylalanyl-L-arginine-p-nitroaniline) at a final concentration of 400 μM.

(f) Activated Protein C (aPC)

Activated Protein C catalytic activity is determined using the chromogenic substrate Spectrozyme PCa (H-D-lysyl(-Cbo)-L-prolyl-L-arginine-p-nitroaniline) at a final concentration of 400 μM (about 4×Kₘ).

(g) Plasmin Assay

Plasmin catalytic activity is determined using the chromogenic substrate S-2366 (L-Pyroglutamyl-L-prolyl-L-arginine-p-nitroaniline) at a final concentration of 300 μM (about 4×Kₘ).

(h) Recombinant Tissue Plasminogen Activator (rt-PA)

rt-PA catalytic activity is determined using the substrate, Pefachrome t-PA (CH₃SO₂-D-hexahydrotyrosine-glycyl-L-arginine-p-nitroaniline) at a final concentration of 500 μM (about 3×Kₘ).

(i) Urokinase

Urokinase catalytic activity is determined using the substrate S-2444 (L-Pyroglutamyl-L-glycyl-L-arginine-p-nitroaniline) at a final concentration of 150 μM (about 7×Kₘ).

(j) Chymotrypsin

Chymotrypsin catalytic activity is determined using the chromogenic substrate, S-2586 (Methoxy-succinyl-L-argininyl-L-prolyl-L-tyrosine-p-nitroaniline) at a final concentration of 100 μM (about 8×Kₘ).

(k) Trypsin

Trypsin catalytic activity is determined using the chromogenic substrate S-2222 (N-benzoyl-L-isoleucyl-L-glutamyl [-methyl ester]-L-arginine-p-nitroaniline) at a final concentration of 300 μM (about 5×Kₘ).

E. Assays for Measuring the Inhibition of the fVIIa/TF Complex by NAP

(a) fVIIa/TF fIX Activation Assay

This Example measures the ability of NAP proteins to act as inhibitors of the catalytic complex of fVIIa/TF, which has a primary role in initiation of the coagulation response in the ex vivo prothrombin time assay (Example 5B). Activation of tritiated fIX by the rFVIIa/rTF/PLV complex is assessed by determining the respective intrinsic inhibition constant, Kᵢ*.

Lyophilized, purified, recombinant human fVlla is reconstituted in HBS (10 mM HEPES, pH 7.5, 150 mM sodium chloride) prior to use. Purified human fX and fXa (free fXa) is activated and prepared from fX as described (Bock, et al. Biochem. Biophys. 273:375-388 (1989)). Active site-blocked human fXa (EGR-FXa), which had been irreversibly inactivated with L-Glutamyl-L-glycyl-L-arginyl chloromethylketone, is also used.

Recombinant human tissue factor (rTF) apoprotein is incorporated into phospholipid vesicles (rTF/PLV), consisting of phosphotidyl choline (75%, w/v) and phosphotidyl serine (25%, w/v) in the presence of detergent, as described by Ruf et al. (Methods in Enzymology 222: 209-224 (1993)). The buffer used for all assays is HBSA, HBS containing 0.1% (w/v) bovine serum albumin.

The activation of human ³H-Factor IX (fIX) by the rfVIIa/rTF complex is monitored by measuring the release of the radiolabelled activation peptide. Purified human fIX is radioactively labeled by reductive tritiation as described (Van Lenten and Ashwell, J. Biol. Chem. 246:1889-1894 (1971)).

The assay for Kᵢ* determinations is conducted as follows: rfVIIa and rTF/PLV are combined in a polypropylene test tube, and allowed to form a complex for 10 min in HBSA, containing 5 mm CaCl₂. Aliquots of rfVIIa/rTF/PLV complex are combined in the appropriate polypropylene microcentrifuge tubes with EGR-fXa or free fXa, when included, and either the NAP protein at various concentrations, after dilution into HBSA, or HBSA alone (as Vₒ (uninhibited velocity) control). Following an incubation of 60 min at ambient temperature, reactions are initiated by the addition of ³H-FIX. The final concentration of the reactants in 420 μl of HBSA is: rfVIIa (50 pM), rTF (2.7 nM), PLV (6.4 μM), either EGR-fXa or free fXa (300 μM), recombinant NAP protein (5-1,500 μM), ³H-fIX (200 nM), and CaCl₂ (5 mM). In addition, a background control reaction is run that included all of the above reactants, except rfVIIa.

At specific time points (8, 16, 24, 32, and 40 min), 80 μl of the reaction mixture is added to an eppendorf tube that contained an equal volume of 50 mM EDTA in HBS with 0.5% BSA to stop the reaction; this is followed by the addition of 160 μl of 6% (w/v) trichloroacetic acid. The protein is precipitated, and separated from the supernatant by centrifugation at 16,000×g for 6 min at 4° C. The radioactivity contained in the resulting supernatant is measured by removing aliquots that are added to Scintiverse BD (Fisher Scientific, Fairlawn, N.J., USA), and quantitated by liquid scintillation counting. The control rate of activation is determined by linear regression analysis of the soluble counts released over time under steady-state conditions, where less than 5% of the tritiated fIX is consumed. The background control (<1.0% of control velocity) is subtracted from all samples. Ratios of inhibited steady-state velocities (V), in the presence of a NAP protein, to the uninhibited control velocity of rfVIIa/TF alone (Vₒ) are plotted against the corresponding concentrations of NAP proteins. These data are then directly fit to an equation for tight-binding inhibitors (Morrison and Walsh, Adv. Enzymol. 61:201-300 (1988)), from which the apparent equilibrium dissociation inhibitory constant Kᵢ* is calculated.

(b) Factor VIIa/Tissue Factor Amidolytic Assay

The ability of NAP proteins to act as an inhibitor of the amidolytic activity of the fVIIa/TF complex is assessed by determining the respective inhibition constant, Kᵢ*, in the presence and absence of active site-blocked human Factor Xa (EGR-fXa).

rFVIIa/rTF amidolytic activity is determined using the chromogenic substrate S-2288 (H-D-isoleucyl-L-prolyl-L-arginine-p-nitroaniline). The substrate is reconstituted in deionized water prior to use. rfVIIa and rTF/PLV are combined in a polypropylene test tube, and allowed to form a complex for 10 min in HBSA, containing 3 mM CaCl₂. The assay for Kᵢ* determinations is conducted by combining in appropriate wells of a microtiter plate 50 μl of the rfVIIa/rTF/PLV complex, 50 μl of EGR-fXa, and 50 μl of either a NAP protein at various concentrations, after dilution into HBSA, or HBSA alone (for Vₒ (uninhibited velocity) measurement). Following an incubation of 30 min at ambient temperature, the reactions are initiated by adding 50 μl of S-2288. The final concentration of reactants in a total volume of 200 μl of HBSA is: recombinant NAP (0.025-25 nM), rfVIIa (750 pM), rTF (3.0 nM), PLV (6.4 μM), EGR-fXa (2.5 nM), and S-2288 (3.0 mM, 3×Kₘ).

The amidolytic activity of rfVIIa/rTF/PLV is measured as a linear increase in the absorbance at 405 nm over 10 min (velocity), using a Thermo Max® Kinetic Microplate Reader (Molecular Devices, Sunnyvale, Calif., USA), under steady-state conditions, where less than 5% of the substrate is consumed. Ratios of inhibited pre-equilibrium, steady-state velocities (Vᵢ), in the presence of NAP protein, to the uninhibited velocity in the presence of free fXa alone (Vₒ) are plotted against the corresponding concentrations of NAP. These data are then directly fit to the same equation for tight-binding inhibitors, used in Example 5E(a), from which the apparent equilibrium dissociation inhibitory constant Kᵢ* is calculated.

1-17. (canceled)

18: A method of treating colorectal cancer in a human subject in need thereof comprising administering rNAPc2/pro to the subject twice-weekly via subcutaneous injection in a dosage range of about 1 to about 10 μg/kg body weight, and subsequently administering FOLFOX to the subject.

19. The method of claim 18, further comprising administering bevacizumab.

20-82. (canceled)

83. A method for treating cancer comprising administering to a subject in need thereof an anti-metastatic agent and an anti-cancer agent.

84. The method of claim 83, wherein the anti-metastatic agent is a NAP protein.

85. The method of claim 83, wherein the cancer is selected from the group consisting of colorectal cancer, melanoma and lung cancer.

86. A method for decreasing or inhibiting tumor metastasis comprising administering to a subject in need thereof an anti-cancer agent and a NAP protein.

87. A method for reducing tumor burden comprising administering to a subject in need thereof an anti-cancer agent and a NAP protein.

88. The method of claim 83, 86 or 87, wherein the anti-cancer agent is selected from the group consisting of a chemotherapeutic agent and a targeted anti-cancer agent.

89. The method of claim 88, wherein the chemotherapeutic agent is FOLFOX or FOLFIRI.

90. The method of claim 88, wherein the targeted anti-cancer agent is bevacizumab.

91. The method of claim 86 or 87, wherein the anti-cancer agent is administered concurrently, prior to, or subsequent to the NAP protein.

92. The method of claim 83, wherein the anti-cancer agent is administered concurrently, prior to, or subsequent to the anti-metastatic agent.

93. The method of claim 84, 86 or 87, wherein the NAP protein is selected from a group consisting of a NAP protein that has a NAP domain of Formula III, rNAPc2, and rNAPc2/pro.

94. The method of claim 84, 86 or 87, wherein the NAP protein is administered subcutaneously.

95. The method of claim 83, 86 or 87, wherein the subject is a mammal.

96. The method of claim 95, wherein the mammal is a human.


SEQUENCE LISTING (20 sequences)

SEQ ID NO: 1 - protein, 77 aa, Ancylostoma caninum
KAYPECGENEWLDDCGTQKPCEAKCNEEPPEEEDPICRSRGCLLPPACVCKDGFYRDTVIGDCVREEECDQHEIIHV

SEQ ID NO: 2 - protein, 75 aa, Ancylostoma caninum
LAYPECGENEWLDVCGTKKPCEAKCSEEEEEDPICRSFSCPGPAACVCEDGFYRDTVIGDCVKEEECDQHEIIHV

SEQ ID NO: 3 - protein, 84 aa, Ancylostoma caninum
KATMQCGENEKYDSCGSKECDKKCKYDGVEEEDDEEPNVPCLVRVCHQDCVCEEGFYRNKDDKCVSAEDCELDNMDFIYPGTRN

SEQ ID NO: 4 - protein, 85 aa, Ancylostoma caninum
KATMQCGENEKYDSCGSKECDKKCKYDGVEEEDDEEPNVPCLVRVCHQDCVCEEGFYRNKDDKCVSAEDCELDNMDFIYPGTRNP

SEQ ID NO: 5 - protein, 7 aa, Ancylostoma caninum
  PEPTIDE (1)..(7): Xaa is any amino acid
XXXXXXX

SEQ ID NO: 6 - protein, 5 aa, Ancylostoma caninum
  PEPTIDE (1)..(5)
EIIHV

SEQ ID NO: 7 - protein, 5 aa, Ancylostoma caninum
  PEPTIDE (1)..(5)
GPYRD

SEQ ID NO: 8 - protein, 5 aa, Ancylostoma caninum
  PEPTIDE (1)..(5)
GFTRN

SEQ ID NO: 9 - protein, 5 aa, Ancylostoma caninum
  PEPTIDE (1)..(5)
GYYRD

SEQ ID NO: 10 - protein, 5 aa, Ancylostoma caninum
  PEPTIDE (1)..(5)
GYYRN

SEQ ID NO: 11 - protein, 5 aa, Ancylostoma caninum
  PEPTIDE (1)..(5)
GLYRD

SEQ ID NO: 12 - protein, 5 aa, Ancylostoma caninum
  PEPTIDE (1)..(5)
DIIMV

SEQ ID NO: 13 - protein, 6 aa, Ancylostoma caninum
  PEPTIDE (1)..(6)
FITFAP

SEQ ID NO: 14 - protein, 5 aa, Ancylostoma caninum
  PEPTIDE (1)..(5)
MEIIT

SEQ ID NO: 15 - protein, 7 aa, Ancylostoma caninum
  PEPTIDE (1)..(7)
  MISC_FEATURE (1)..(2): Xaa = any amino acid provided at least one Xaa at location 1 or 2 is Glu or Asp
XXGPYRD

SEQ ID NO: 16 - protein, 7 aa, Ancylostoma caninum
  PEPTIDE (1)..(7)
  MISC_FEATURE (1)..(2): Xaa = any amino acid provided at least  one Xaa at position 1 or 2 is Glu or Asp
XXGFYRN

SEQ ID NO: 17 - protein, 7 aa, Ancylostoma caninum
  PEPTIDE (1)..(7)
  MISC_FEATURE (1)..(2): Xaa = any amino acid provided at least  one Xaa at position 1 or 2 is Glu or Asp
XXGYYRD

SEQ ID NO: 18 - protein, 7 aa, Ancylostoma caninum
  PEPTIDE (1)..(7)
  MISC_FEATURE (1)..(2): Xaa = any amino acid provided at least  one Xaa at position 1 or 2 is Glu or Asp
XXGYYRN

SEQ ID NO: 19 - protein, 7 aa, Ancylostoma caninum
  PEPTIDE (1)..(7)
  MISC_FEATURE (1)..(2): Xaa = any amino acid provided at least  one Xaa at position 1 or 2 is Glu or Asp
XXGLYRD

SEQ ID NO: 20 - protein, 4 aa, Ancylostoma caninum
  PEPTIDE (1)..(4)
  MISC_FEATURE (1)..(4): Xaa = any amino acid
XXXX